(19) United States
(12) Patent Application Publication
(10) Pub. No.: US 2023/0240989 A1
(43) Pub. Date: Aug. 3, 2023

(54) LIPOSOMAL OZONE NANOSOLUTIONS

(71) Applicant: Ahmet Ümit SABANCI, Osmangazi, Bursa (TR)

(72) Inventor: Ahmet Ümit SABANCI, Osmangazi, Bursa (TR)

(21) Appl. No.: 18/004,973

(22) PCT Filed: Mar. 1, 2022
(86) PCT No.: PCT/TR2022/050177

§ 371 Date: Jan. 10, 2023

(30) Foreign Application Priority Data
Mar. 1, 2021 (TR) 2021/004032

(51) Int. Cl. A61K 9/127; A01N 25/04; A61K 33/00; A01N 59/00; A01P 7/02; A61P 31/04; A61K 9/00; A61P 31/12; A01P 3/00; A61K 45/06

(52) U.S. Cl. CPC A61K 9/127; A01N 25/04; A61K 33/00; A01N 59/00; A01P 7/02; A61P 31/04; A61K 9/0019; A61P 31/12; A01P 3/00; A61K 45/06

(57) ABSTRACT

Disclosed are liposomal ozone nanosolutions with antiviral, antifungal and antibacterial effects; wound healing, tissue healing properties suitable for use in human, veterinary, food, agriculture and chemistry and cosmetics fields. The liposomal ozone nanosolution can be used as nasal spray, mouth spray, ear drops, eye drops, hand and face disinfectant, vaginal, intravesical, rectal solutions, intraarticular, subcutaneous, intramuscular, intravenous injection solutions for human and veterinary hygiene purposes and can be used as a surface cleaner as well. The effectiveness of the liposomal ozone nanosolutions has been proven on viruses such as influenza or corona virus, especially Sars-Cov-2 and bacteria, especially Staphylococcus aureus and Escherichia coli.

### FIELD OF THE INVENTION

The present invention relates to liposomal ozone nanosolutions with antiviral, antifungal and antibacterial effects suitable for use in human, veterinary, food, agriculture and chemistry.

### STATE OF THE ART

The use of nanoparticles has become widespread in the chemical industry in recent years. Efficiency in nano sizes is achieved with smaller particles, and activities on viruses and bacteria increase with the help of these technologies. Simultaneously, active drugs can be transported to organelles such as mitochondria within the cell and more effective treatments can be provided with less chemicals. At the same time, the use of nano-sized products in other sectors of the chemical industry provides advantages. For example, disinfectant products made with nanoparticles form a film in the application area and cover the environment and it is possible to provide a longer effect with a small amount of product. Another particle system made for this purpose is the formation of liposomes. Liposomes are made by breaking down lipids with ultrasonic fat breakdown devices or similar nanotechnological methods. Drugs and active substances can be transmitted in these liposomes and the transmission of active substances into cells becomes easier. Normally, it is not possible for the active substances to enter the cancer cells, however it is possible to introduce the active substance into the cancer cell in the liposome. Developments continue in the technical field regarding the use of liposomes and nanocarriers created with oils in the fields of medicine, food, agriculture, veterinary medicine, cosmetics and disinfection.

For example, patent EP1746976B1 provides liposome compositions containing substituted ammonium and/or polyanion and optionally a desired therapeutic or imaging entity. TR97/01683 national patent application relates to the development of liposomal drug delivery systems for the biodistribution of cyclosporine A or similar hydrophobic drugs in body fluids and/or tissues.

Methods of use of ozone in gas form have been widely used for many years such as medical field, disinfection, antibacterial activity, repellent effect, insecticide effects, cosmetic effects, food preservative effects, agriculture, and veterinary fields. However, these methods make it difficult for ozone gas to become widespread, as it is unstable and has a half-life of 20 minutes. In recent years, the transportation of ozone with water has started to become widespread in methods such as disinfection of water. However, these methods were not enough to generalize the use of ozone. Studies are carried out in different fields on the use of oils as ozone carriers so as to solve these problems. However, problems such as stable ozone transport and release capacities of oils and the lack of effective particle size optimization according to the application area are encountered in current applications.

As a result, due to the abovementioned disadvantages and the insufficiency of the current solutions regarding the subject matter, a development is required to be made in the relevant technical field.

### BRIEF DESCRIPTION OF THE INVENTION

The present invention relates to liposomal ozone nanosolutions which fulfill the abovementioned requirements, eliminate all disadvantages and bring some additional advantages.

Another aim of the invention is to produce antiviral, antibacterial, antifungal insecticide, acaricide effective, wound healing tissue healer, plant developer, plant protector, bee treatment, liposomal ozone nanosolutions suitable for use in human, veterinary, agriculture and chemistry fields, to contribute to public health on a global scale.

Another aim of the invention is to present liposomal ozone nanosolutions in formulations with proven stability, whose nanoparticle size distribution, effective ozone dose and ozone release profile can be determined for the field of use and type of application.

An aim of the invention is to present liposomal ozone nanosolutions with both anti-viral and anti-bacterial activity; non-toxic effect; no negative effect on the viability of healthy cells; that have both regenerative and proliferative effects on human respiratory system (tracheo-bronchial) cells.

Another aim of the invention is to present liposomal ozone nanosolutions that offer stable antibacterial activity on bacteria such as Staphylococcus aureus or Escherichia coli and stable antiviral activity on viruses such as Sars-Cov-2.

Another aim of the invention is to present liposomal ozone and nanocarrier polymer nanosolutions that do not have irritant or hyperreactive effects on skin, mucosal tissues or ocular, vaginal, rectal areas.

Another aim of the invention is to present liposomal ozone nanosolutions that do not have cytotoxic effects in mucosal tissues.

Another aim of the invention is to present products suitable for use as nasal spray, mouth spray, ear drops, eye drops, vaginal solution, rectal solution, intraperitoneal solution, bladder solution, hand and face disinfectant, and surface cleaner for personal hygiene, to be used in the prevention and healing of viral, bacterial or fungal diseases.

Another aim of the invention is to present antiviral, antibacterial, antiparasitic, antifungal effective barrier solutions on skin and surfaces, suitable for use in human, veterinary, agriculture and chemistry fields containing liposomal ozone nanosolutions.

Another aim of the invention is to present nasal spray, mouth spray, ear drops, eye drops, vaginal solution, rectal solution, intraperitoneal solution, intravesical solution, intravenous and intraarticular/subcutaneous/intramuscular injection solutions with antiviral, antibacterial, antifungal effect, wound healing tissue healing effect for human, veterinary use containing liposomal ozone nanosolutions.

In order to fulfill the above-described aims, the invention mainly consists of nanomaterials obtained from ozonated emulsifier, and also contains nanoparticle liposomes obtained from ozonated oil providing synergetic effect.

The structural and characteristic features of the present invention will be understood clearly by the following detailed description and therefore the evaluation shall be made by taking the detailed description into consideration.

### DESCRIPTION OF THE FIGURES

FIG. 1, the size distribution graph of the nanosolution obtained from ozonated oil is given according to the density. [Density (Percent)×Size (d·nm)]

FIG. 2, the size distribution graph according to the density of the nanosolution obtained from the ozonized emulsifier is given. [Density (Percent)×Size (d·nm)]

FIG. 3 is a coating of ozonated surfactant (emulsifier polysorbates) glycerin and PEG 400. [Density (Percent)×Size (d·nm)]

FIG. 4 is coating with ozonated surfactant and non-ozone surfactant. [Density (Percent)×Size (d·nm)]

FIG. 5, fruit height and diameter measurements of tomatoes are given.

FIG. 6 shows the graph of color determination in tomato.

FIG. 7, SCKM values in tomato are given.

FIG. 8 gives TEA values in tomato.

FIG. 9 gives the yield per plant in tomato.

FIG. 10, leaf and root lengths of leaf lettuce are given.

FIG. 11, root wet and dry weight of the leaf lettuce is given.

FIG. 12, leaf wet and dry weight of the leaf lettuce is given.

FIG. 13, the number of marketable and non-marketable leaves is given.

FIG. 14, leaf proportional water content of leaf lettuce is given.

FIG. 15, the total amount of chlorophyll in the leaf in the leaf lettuce is given.

FIG. 16, leaf color of leaf lettuce is given.

### DETAILED DESCRIPTION OF THE INVENTION

In this detailed description, the preferred embodiments of the inventive nanosolutions are described only for clarifying the subject matter in a manner such that no limiting effect is created.

In the state of the art, obtaining nanoparticles in sizes that will allow the penetration of ozone into application areas such as cells and the release rates of ozone provided by these particles were limited in systems where ozone is carried only by oils. Although in theory these nanoparticles appear to cover all nano sizes down to micron sizes, in practice, it was not possible to obtain stable particles of nano size, such as below 50 nm, that would allow penetration into the cell. Studies have been carried out with the invention so as to provide stable systems containing the smallest nanoparticles that can carry ozone in order to increase the application efficiency of ozone. Surprisingly, these studies have shown that substances such as Polysorbates and PEG, which are included in the emulsifier group, can be ozonized and form stable nanoparticles in smaller sizes compared to carrier oils.

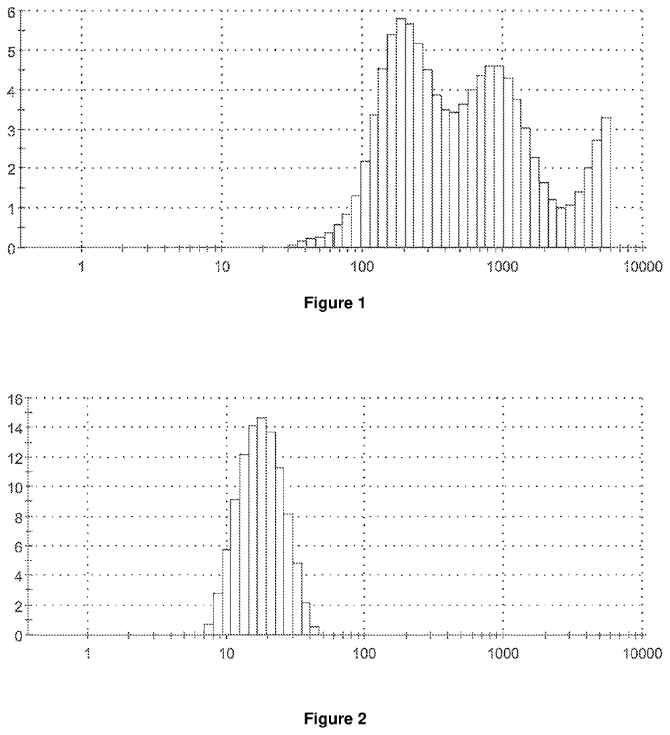

Size distribution analysis was carried out only in nanosolutions obtained from ozonated oil in the studies carried out within the scope of the invention and the graph of size distribution according to density is given in FIG. 1. Liposomal nanosolutions made with ozonized oil containing 10% ozone gas by weight, ranged between 30 nm and 6000 nm, and at most concentrated solutions of 200 and 1000 nanometers could be formed. Dimensional analysis results are shown in Chart 1 below.

Chart 1. Size distribution analysis results according to density in
nanosolution obtained from ozonated oil (Dispersant RI 1.330
Sample RI 1, Viscosity 0.8872 cP, 25 C,)

| | | | | | Standard |
|---|---|---|---|---|---|
| | | | Dimension | % | Deviation |
| | | | (d · nm) | Intensity: | (d · nm) |
| Z- Mean(d · nm) | 363.1 | Peak 1 | 228.8 | 49.0 | 106.8 |
| Pdl | 0.574 | Peak 2 | 1051 | 40.0 | 524.4 |
| Cutting point | 0.926 | Peak 3 | 4404 | 11.0 | 969.5 |

In the studies, size distribution analysis was also carried out for the nanosolution obtained from only ozonated emulsifier, the graph of the size distribution according to the density is given in FIG. 2. In this example, the Polysorbate 80 emulsifier was ozonized at 3% by weight. It has been observed that ozonated polysorbate 80 concentrate can form nanoparticles up to 16 nm in size. It has been observed that nanoparticles up to 7 nm in size can be formed in the nanosolution obtained from ozonated polysorbate 80 with water. In the size distribution analysis, nanoparticles with a distribution between 7 nm and 50 nm were obtained, as seen in FIG. 2. Since nanoparticles of this size can transmit ozone into the cell, for example, they will have an antiviral effect on viruses. At the same time, it will increase the energy generation potential of the cell by entering the mitochondria inside the cell and will activate the intracellular repair mechanism by inducing intracellular growth factors.

Chart 2. Size distribution analysis results according to density in
nanosolution obtained from ozonated emulgator (Dispersant RI 1.330
Sample RI 1, Viscosity 0.8872 cP, 25 C,)

| | | | | | Standard |
|---|---|---|---|---|---|
| | | | Dimension | % | Deviation |
| | | | (d · nm) | Intensity: | (d · nm) |
| Z- Mean (d · nm) | 16.52 | Peak 1 | 19.03 | 100.0 | 7.017 |
| Pdl | 0.125 | Peak 2 | 0.000 | 0.000 | 0.000 |
| Cutting point | 0.946 | Peak 3 | 0.000 | 0.000 | 0.000 |

In the studies carried out within the scope of the invention, it has been demonstrated that alternative formulations in which the nanoparticle size distribution and ozone emission velocity distribution can be determined, can be developed with a nanotechnological method suitable for the content, in terms of the application area of ozonated emulsifier and liposomal ozone nanosolutions obtained from ozonated oils. Nanoparticles obtained by ozonation of emulsifiers provide relatively smaller and faster ozone release to oils. Ozonated liposomal nanosolutions, which are formed by the breakdown of ozonated oils by nanotechnological methods, create many areas of use due to their stability and long-lasting effectiveness. In solutions made with ozonated oil, ozonated emulsifier, and water, the ozone ratio in the oil is determined and solutions are created with the same standards. For example, a solution with a high oil content is used for a long-acting solution, and ozone gas is kept in the oil at a rate of 1-5% by weight. In order to create shorter-acting products, solutions containing 6-15% by weight ozone gas are created. In this way, the effectiveness of liposomal ozone nanosolutions obtained according to the content has been proven by scientific studies in the fields of human, veterinary, food and agriculture; its use in formulations for these areas is also within the scope of the invention.

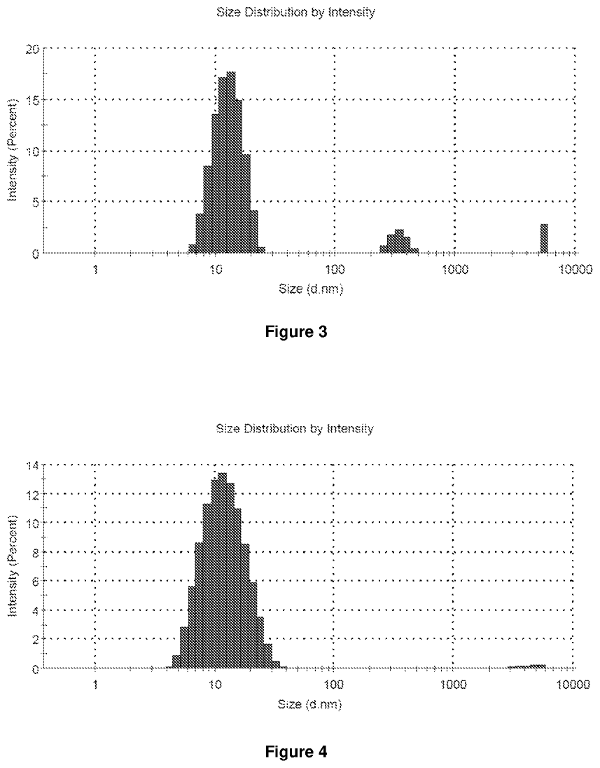

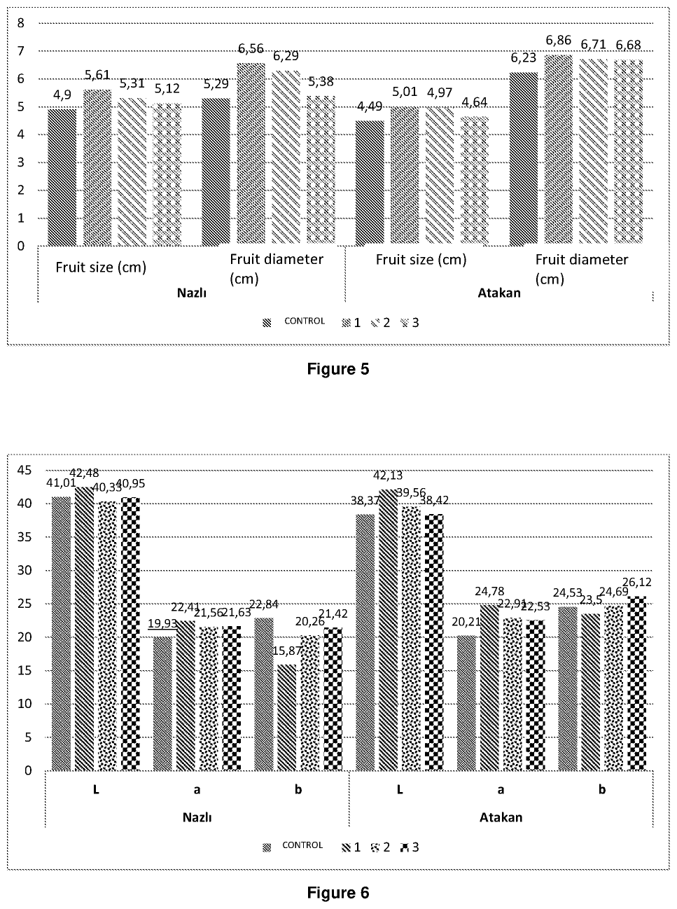

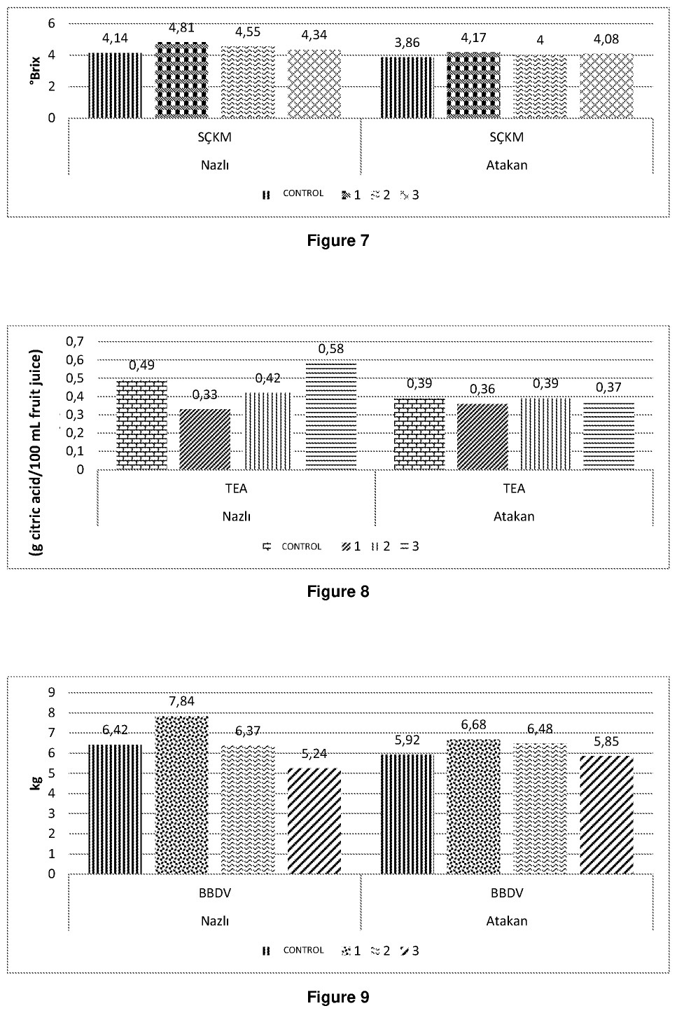

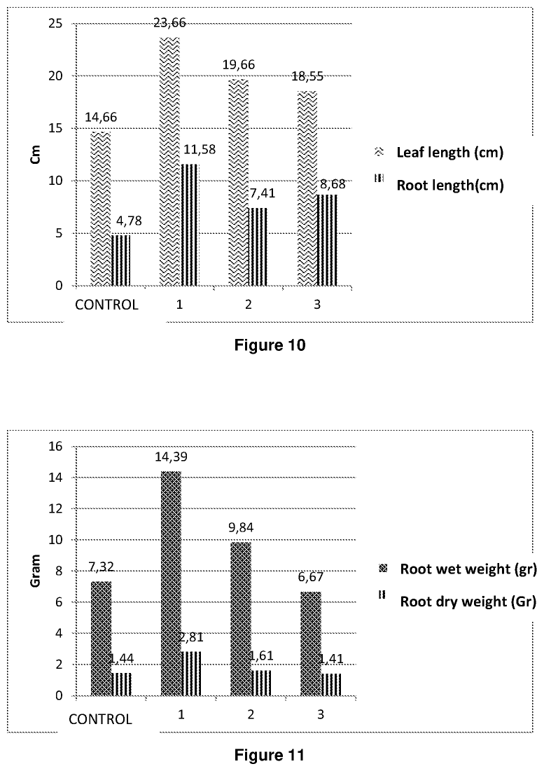

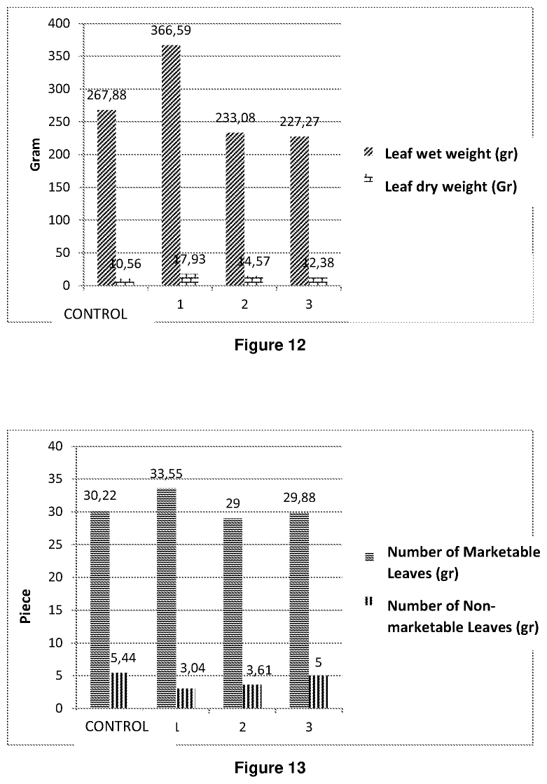

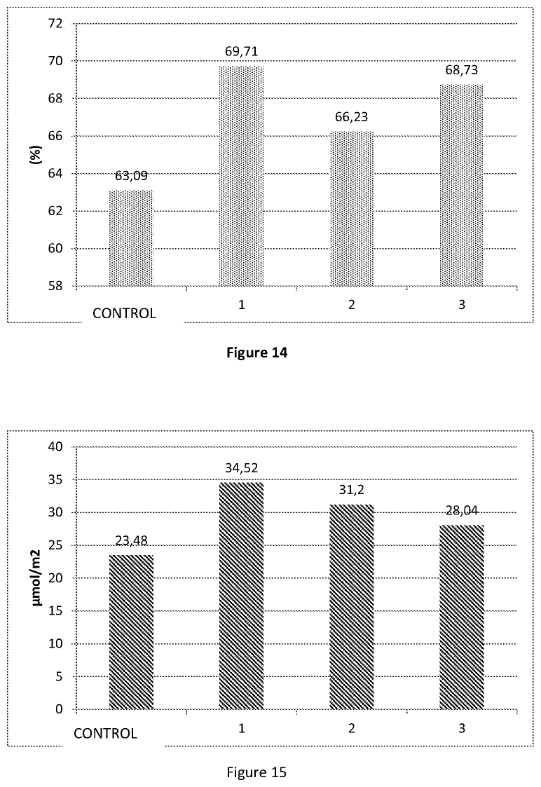

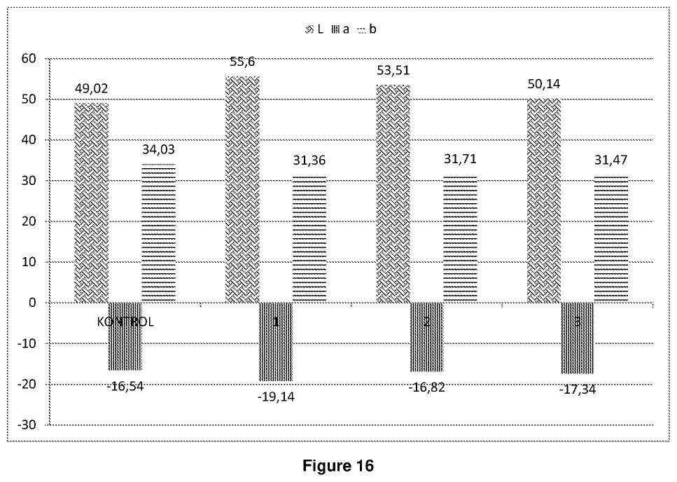

Since the pure structures formed by ozone emulsifiers are much smaller particles than liposomes, they reveal more active effects. These structures have active antibacterial effects and at the same time, it is important that they are be coated so as to provide slower released effects. Slower release properties are created by coating the ozone emulsifier with glycerin and PEG. FIG. 3 shows the emulsifier liposome.

The smallest liposome structure formed by coating emulsifiers with emulsifiers is called niosome. The smallest niosome structure is formed by coating the ozone emulsifier with non-ozone emulsifiers. In this way, it is possible to obtain niosomes with active and slow-release ozone. In FIG. 4, ozonated polysorbate is again coated with polysorbate.

At the same time, these niosomes can be coated with polymers such as glycerin, peg, chitosan, mannitol, and dextran. One or more of these coatings can be used, especially in intravenous formulations and regional injections, so as to reduce the burning effect of ozone and to ensure that it reaches the target organ more easily.

The invention described above is basically liposomal ozone nanosolutions containing ozonated emulsifier.

The “emulsifier” mentioned in the invention is individuals or combinations selected from lecithin, lysophospholipid, polyethylene glycol (PEG), phosphatidylethanolamine (PEG-PE), pluronic, polysorbates (polysorbate 20, polysorbate 80, etc.), or a pharmaceutically acceptable emulsifier.

In preferred embodiments of the invention, the emulsifier mentioned is a polysorbate or PEG.

The “ozone gas” mentioned in the invention is an allotrope of oxygen (O₃), a colorless gas with molecules consisting of 3 oxygen atoms and is found in the upper layers of the atmosphere in nature. Under normal conditions, the amount of ozone in the lower parts of the atmosphere is about 0.04 ppm.

A preferred embodiment of the invention also contains water. “Water” mentioned in the invention are individuals or combinations selected from distilled water, salt water (NaCl—H₂O), sugar water, mineral water, deionized water, demineralized water, spring water, saline solution, physiological saline, and plant waters.

A preferred embodiment of the invention also includes ozonated oil obtained by passing ozone gas through carrier oil. The “carrier oil” mentioned in the invention is individuals or combinations selected from soybean oil, centaury oil, sesame oil, palm oil, poppy oil, soy lecithin, cholesterol, b-sterol, triglyceride, olive oil, fish oil, sunflower oil, castor oil, saffron oil, coconut oil, triglyceride derivatives, tributyrin, tricaproin, tricaprylin with paraffin, ethyl oleate, methyl oleate.

A preferred application of the invention also contains at least functional oil. “Functional oil” mentioned in the invention is individuals or combinations selected from fixed and/or essential vegetable oils. The fixed oils mentioned here are vegetable fixed oils such as coconut oil, almond oil, jojoba oil, rosehip seed oil, avocado oil, sesame oil, apricot seed oil, coconut oil, olive oil, sunflower oil, soybean oil. The essential oils mentioned here are essential oils such as sage oil, anise oil, calendula oil, rosemary oil, pine turpentine, cypress oil, tea tree oil, evening primrose oil, bay leaf oil, basil oil, rose oil, borage oil, black pepper oil, clove oil, thyme oil, cumin oil, coriander oil, lavender oil, lemon oil, lemon balm oil, violet oil, myrtle oil, peppermint oil, eucalyptus oil, chamomile oil, orange oil, grapefruit oil, nettle oil, fennel oil, sandalwood oil, garlic oil, cypress oil, jasmine oil, ylang ylang oil, geranium oil, patchouli oil, ginger oil.

A preferred application of the invention also contains at least one organic acid. “Organic acid” mentioned in the invention are individuals or combinations selected from among formic acid, phosphoric acid, hydrochloric acid, acetic acid, propionic acid, butyric acid, valeric acid, caproic acid, oxalic acid, lactic acid, malic acid, citric acid, benzoic acid, carbonic acid, phenol, uric acid, taurine, aminomethylphosphonic acid.

A preferred application of the invention also contains at least an excipient. “Excipients” mentioned in the invention are individuals or combinations selected from among anesthetics, pharmaceuticals pharmaceutical active substances such as water and/or fat soluble vitamins, minerals, hyaluronic acid, thymol, menthol, glycerin, ethyl alcohol, cetyl alcohol, butyl alcohol, benzyl alcohol, amino acids, acetyl cysteine, glutathione, herbal extracts, lidocaine, xylocaine.

Preferred applications of the invention consist of nanoparticle liposomes containing ozonated emulsifier and/or ozonated carrier oil, glycerin, hyaluronic acid, menthol, distilled water, NaCl composition.

The embodiments of the invention include 10 stock ppm-60 000 stock ppm ozone gas.

The embodiments of the invention contain stable active ozone gas at doses of 100 ppm, 200 ppm, 500 ppm, 1000 ppm, 1600 ppm, 2000 ppm, 3000 ppm, depending on the application area.

Preferred embodiments of the invention include liposomal ozone nanoparticles in sizes below 1000 nm, preferably below 200 nm, more preferably below 50 nm.

According to an embodiment of the invention, the method of forming liposomal ozone nanosolutions includes the following steps:
Selecting the appropriate emulsifier and/or carrier oil for the application purpose,
weighing the emulsifier and/or carrier oil,
ozonation of emulsifier and/or carrier oil by passing ozone gas or nanobubble ozonated water through it,
weighting ozonated emulsifier and/or carrier oil again and determining the ozone content,
coating of ozonated emulsifier and/or ozonated/non-ozone oil with glycerin and/or PEG.
reduction of ozonated emulsifier and/or carrier oil to nanosize, preferably with water, by shredding for at least 1 hour with a min. 12000-revolution mixer,
preferably adding and mixing components selected from at least one excipient, at least one functional oil, at least one organic acid.

### Example 1

An example of the invention consists of ozonated emulsifier. The emulsifier chosen for the production of this exemplary composition is weighed. It is ozonized by passing ozone gas through the emulsifier, the amount of which is determined. Ozonation processes are carried out with ozone micro/nano-bubble generators. The ozonized emulsifier is weighed again, and the amount of ozone gas absorbed in it is measured by weight. Nanoparticle sizes containing ozone nano-bubble can be reduced to the range of 5-50 nm by nanotechnological methods. In Table 1, the content information of an example of the invention obtained by this method is given. Accordingly, emulsifiers containing 10-50 000 stock ppm ozone gas are obtained. The preferred ozonated emulsifier herein is a polysorbate or PEG. These nanosolution concentrates preferably contain 10 000 stock ppm of ozone gas.

TABLE 1
Sample content of the invention

| | Preferred amount | Usable amount by weight |
|---|---|---|
| Content | by weight (%) | (%) |
| Emulsifier | 99 | 95-99 |
| Ozone gas | 1 | 0.01-5 |

### Example 2

Another example of the invention is ozonated nanosolution obtained with water from ozonated emulsifiers. A nanosolution is obtained by mixing the ozonated emulsifier, whose ozone amount is determined as above, with the required amount of water with blade mixers with at least 12000 rpm. Nanoparticle sizes containing ozone nano-bubble can be reduced to the range of 5-50 nm by nanotechnological methods. In alternative methods, first the water is ozonated and the emulsifier ozonated water is mixed. In Table 2, the content information of an example of the invention obtained by said methods is given. Accordingly, nanosolutions containing 100-35 000 stock ppm ozone gas are obtained. The preferred ozonated emulsifier herein is a polysorbate or PEG. These examples may preferably contain at least one excipient described above. These nanosolution concentrates preferably contain 500 stock ppm of ozone gas.

TABLE 2
Sample content of the invention

| | Preferred amount | Usable amount by weight |
|---|---|---|
| Content | by weight (%) | (%) |
| Emulsifier | 50 | 1-70 |
| Ozone gas | 0.5 | 0.01-3.5 |
| Water | 49.5 | 23-98.9 |

### Example 3

Another example of the invention is the nanosolution obtained with ozonated carrier oil and water from ozonated emulsifiers. In this example, first of all, the amount of emulsifier is determined, ozonized and weighed to determine the ozone content. On the other hand, the selected carrier oils are ozonated by determining their amounts and weighed to determine the ozone content. The obtained ozonated emulsifier, ozonated carrier oil is mixed with the required amount of water with blade mixers with at least 12000 revolutions to obtain a nanosolution. Ultrasonic cavitation can be used to reduce the size of liposomes containing ozone nano-bubbles to the desired range by nanotechnological methods. Liposomal nanoparticle sizes containing ozone nano-bubble can be reduced to the range of 5-50 nm by nanotechnological methods. In Table 3, the content information of an example of the invention obtained by said methods is given. Accordingly, nanosolutions containing 100-45 000 stock ppm ozone gas are obtained. The preferred example of ozonated emulsifier herein is ozonated polysorbate or PEG, and ozonated carrier oil is sunflower oil or olive oil. Preferably, thymol, orange oil, clove oil, lemon oil can be added. These examples may preferably contain at least one excipient described above. These nanosolution concentrates preferably contain 1100 stock ppm of ozone gas.

TABLE 3
Sample content of the invention

| | Preferred amount by | Usable amount by weight |
|---|---|---|
| Content | weight (%) | (%) |
| Carrier oil | 10 | 0.1-30 |
| Ozone gas | 0.11 | 0.01-4.5 |
| Emulsifier | 10 | 0.1-30 |
| Water | 97.9 | 39-99.79 |

### Example 4

Another example of the invention is ozonated liposomal nanosolutions obtained with ozonated carrier oil from ozonated emulsifiers, and at least one organic acid. In this example, first of all, the amount of emulsifier is determined, ozonized and weighed to determine the ozone content. On the other hand, the selected carrier oils are ozonated by determining their amounts and weighed to determine the ozone content. Nanosolution is obtained by mixing the ozonized emulsifier, ozonated carrier oil with the required amount of water and selected organic acid with blade mixers with at least 12000 rpm. Ultrasonic cavitation can be used so as to reduce particle sizes. In Table 4, the content information of an example of the invention obtained by said methods is given. Accordingly, nanosolutions containing 10 000-60 000 stock ppm ozone gas are obtained. If the preferred ozonated polysorbate or PEG herein is ozonated carrier oil, it is a vegetable oil preferably sunflower oil, olive oil, soybean oil, thymol, orange oil, clove oil, lavender oil, nettle oil, lemon oil, if it is organic acid; it is preferably 100% acetic acid. These examples may preferably contain at least one excipient described above. These nanosolution concentrates preferably contain 30000 stock ppm of ozone gas.

TABLE 4
Sample content of the invention

| | Preferred amount by | Usable amount by weight |
|---|---|---|
| Content | weight (%) | (%) |
| Carrier oil | 30 | 20-40 |
| Ozone gas | 3 | 1-6 |
| emulsifier | 33 | 20-40 |
| Organic acid | 34 | 20-40 |

### Example 5

Another example of the invention is ozonated liposomal nanosolutions obtained in situ with ozonated carrier oil, at least one organic acid and at least one functional oil from ozonated emulsifiers. In exemplary applications of the invention, ozonated oil, ozonated emulsifier and organic acid and functional oils are presented as a composition without mixing thereof. They stand in separate layers. Nanoparticles are formed as a result of mixing them with mixer and water by throwing them into water boilers in agriculture. The aim here is to make carrying easier. For example, 1-6 kilos of stock composition can be thrown into 1 ton of water and spraying can be done on the field. In Table 5, the content information of an example of the invention obtained by said methods is given. Accordingly, nanosolutions containing 10 000-60 000 stock ppm ozone gas are obtained. If the preferred ozonated polysorbate or PEG herein is ozonated carrier oil, it is a vegetable oil preferably sunflower oil, olive oil, soybean oil, functional oil preferably oil, thymol, orange oil, clove oil, lavender oil, nettle oil, if it is organic acid, it is preferably acetic acid. These samples preferably contain at least one excipient described above. These nanosolutions preferably contain 30 000 stock ppm of ozone gas.

TABLE 5
Sample content of the invention

| | Preferred amount by | Usable amount by weight |
|---|---|---|
| Content | weight (%) | (%) |
| Carrier oil | 27 | 20-40 |
| Ozone gas | 3 | 1-6 |
| Emulsifier | 30 | 20-40 |
| Organic acid | 30 | 20-40 |
| Functional oil | 10 | 5-20 |

### Example 6

Another example of the invention is ozonated liposomal nanosolutions obtained from ozonated emulsifiers with ozonated carrier oil, at least one organic acid, at least one functional oil and alcohol. In this example, first of all, the amount of emulsifier is determined, ozonized and weighed to determine the ozone content. On the other hand, the selected carrier oils are ozonated by determining their amounts and weighed to determine the ozone content. Nanosolution is obtained by mixing the ozonized emulsifier, ozonated carrier oil with the required amount of water, selected organic acid, functional oil, and alcohol with blade mixers with at least 12000 rpm. In Table 6, the content information of an example of the invention obtained by said methods is given. Accordingly, nanosolutions containing 10 000-60 000 stock ppm ozone gas are obtained. Preferred herein are ozonated emulsifier polysorbate or PEG, ozonated carrier oil preferably sunflower oil, olive oil and/or soybean oil; the preferred organic acid is 100% acetic acid, and the alcohol is 100% pure vegetable alcohol; the preferred functional oil is thyme oil, orange oil, clove oil, lavender oil, nettle oil and/or lemon oil. These examples may preferably contain at least one excipient described above. These nanosolution concentrates preferably contain 30000 stock ppm of ozone gas.

TABLE 6
Contents of an example of the invention

| | Preferred amount by | Usable amount by weight |
|---|---|---|
| Content | weight (%) | (%) |
| Carrier oil | 27 | 20-40 |
| Ozone gas | 3 | 1-6 |
| Emulsifier | 20 | 10-40 |
| Organic acid | 30 | 20-40 |
| Functional oil | 10 | 5-15 |
| Alcohol | 10 | 5-15 |

The antibacterial, antivirus, antifungal, antiparasitic wound healing and tissue healing properties of the liposomal ozone nanosolutions described above have been proven by the following scientific analyzes. The barrier solution of the invention for barrier formation and applications for intraarticular/subcutaneous/intramuscular injection are suitable for use in the fields of human, veterinary, food, agriculture and chemistry, examples of which are as follows:
as a disinfectant, preservative or food supplement in the food industry;
as a disinfectant, repellent, feed additive in the livestock sector;
use in the health sector, especially in dermatology, ear-nose-throat, eye diseases, oral-dental diseases, digestive system, urology, gynecology, orthopedics, circulatory system diseases, in therapeutic pharmaceutical compositions in gastroenterology, ozone therapy and mesotherapy,
In treatment areas equivalent to treatments with gas ozone,
in the cosmetics industry, especially in the production of personal care products used for skin, mouth and hair care;
in the production of detergents and disinfectants in the chemical industry and as an additive for petroleum products;
as a bleach, disinfectant, antibacterial textile chemical in the textile industry;
preservative and repellent in final products in the agricultural sector, insecticide, pesticide, fungicide, virucidal, bactericide, plant and soil disinfectant in cultivated products;

The antiviral activity mentioned within the scope of the invention may comprise the following viral classes such as Herpesviridae, Hepadnaviridae, HIV, Togaviridae, Arenaviridae, Flaviviridae, Orthomyxoviridae (Influenzavirus A, influenzavirus B, influenzavirus C, isavirus, thogotovirus), Paramyxoviridae, Bunyaviridae, Rhabdoviridae, Filoviridae, Coronaviridae (Corona virus, in particular Sars-Cov-2), Bornaviridae, Arteriviridae and Retro viridae.

The antibacterial activity mentioned within the scope of the invention may comprises bacterial classes such as; Aeromonas hydrophila, Arcanobacterium pyogene, Bacillus thurgiensis, Bacillus anthracis, Bacillus cereus, Clostridium botulinum, Clostridium perfringens, Clostridium septicum, Clostridium sordellii, Clostridium tetani, Corynebacterium diphtheriae, Escherichia coli, Listeria monocytogenes, Pseudomonas aeruginosa, Staphylococcus aureus, Streptococcus pneumonia, Streptococcus pyogenes intermedius or Vibrio cholera. Other bacterial infections considered are eye, ear, nose, throat, vaginal, rectal infections.

The following products containing liposomal ozone nanosolutions of the invention in their formulations for use in the areas described above are within the scope of the invention:
Nose, eye, ear, throat solution, vaginal, rectal solutions, drops or sprays formulated for human or veterinary use. 500-3000 ppm
For subcutaneous, intraarticular, intramuscular, intravenous, intraarticular, intravesical, intraperitoneal mesotherapy applications, ready-made glass syringes due to ozone interaction, silicone caps, glass vial, silicone caps, ampoules, bottles with vaginal rectal apparatus, injectable solutions delivered in ozone resistant serum bottles and plastic bags. 500-3000 ppm
Hair solutions containing combined vitamins, minerals, and active ingredients such as minoxidil and finasteride for hair growth and new hair growth. 500-2000 ppm
Bee solutions for use in beekeeping treatments. (2000 ppm liposomal ozone nanosolution+thymol+sugar water mixture)
It is a fruit and vegetable solution used in dried fruits and vegetables, fresh fruits and vegetables and meats to increase the shelf life with its oil content, to be stored for a long time and to reduce the harm of pesticides or to reduce the use of pesticides to make them look shiny. Liposomal ozone nanosolution containing glycerin, ozonated sunflower and/or ozonated polysorbate, containing 1000-2000 ppm ozone as insecticide, antiparasitic and antibacterial.
Suitable skin solution for dermatitis treatments in allergic diseases. Ozone emulsifier and oil mixture, or ozone emulsifier water mixture containing 500-10000 ppm ozone
Serum solution for use in parenteral injections. Ozone emulsifier water mixture or ozone emulsifier, ozonated oil and water containing 1-6 mg/kg 200 ppm-2000 ppm ozone.
Injectable solution containing active ingredients such as combined fat-soluble vitamins, hyaluronic acid, amino acids and minerals, anesthetic agents such as lidocaine, for subcutaneous, intramuscular, intraarticular mesotherapy. It contains 300-2000 ppm of ozone.
Washing solution used during surgery in the abdomen and other cavities. It contains 300-2000 ppm of ozone.
Solution presented in spray form for wound treatment, combined with disinfectants containing vitamins, minerals or iodine, zinc, betadine, silver ions. Ozone emulsifier water or ozone emulsifier and ozone oil water containing 500-10000 ppm ozone.
Solutions or creams presented in spray forms in combination with other skin rejuvenating and spot lightening products for skin rejuvenation. Ozone emulsifier water or ozone emulsifier and ozone oil water containing 500-40000 ppm ozone.
Solution for presentation in urology as an injector for an antiseptic probe or as a lubricant in one-shot silicone. Ozone emulsifier water or ozone emulsifier ozonated oil water containing 100-2000 ppm ozone.
Glass injection for antibacterial bladder in urology, solution to be presented in ozone resistant puar forms. It contains 300-2000 ppm ozone.
Solution for vaginal antisepsis presented in the form of disposable packages, sachets or injectors. It contains 1000-4000 ppm ozone.
Injectable solution offered in injection form for oviducts for infertility purposes. It contains 300-2000 ppm of ozone.
Injectable solution offered in antiseptic spray, ampoule, injection and vial forms for intracavity and root canal treatment in dental health. It contains 300-2000 ppm of ozone.
It is a solution containing ozone emulsifier, ozone emulsifier and oil-water in capsule or gel forms that dissolve in the stomach, dissolve in the small intestine, melt in the large intestine for the health of the digestive system. It contains 10-200 ppm ozone.
It is one-liter or disposable rectal solution to be used in large intestine diseases. It contains 100-2000 ppm ozone.
Suppositories solidified with solid paraffin or witepsol for use in large intestine diseases. It contains 2000-10000 ppm ozone.
It is a toothpaste for oral health, a spray, a mouthwash and preferably a lozenge containing sweetener.
Solution for use as a feed additive in animal health. Injectable solution offered in syringe, vial, ampoule forms for intramuscular, intra-articular, intravenous, subcutaneous, intravaginal, intraperitoneal injection for the treatment of infection in order to protect animal health. It contains 300-3000 ppm ozone.
Liter or single use rectal solution to protect animal health. It contains 300-3000 ppm ozone.
Suppositories solidified with solid paraffin or witepsol to protect animal health. It contains 2000-10000 ppm ozone.
Solution for use as a repellent, insecticide and disinfectant in environments such as chicken farms, barns and pens. It contains 500-10000 ppm ozone.
Hygiene product presented in the form of spray or solution for wound and skin care, disinfection, hygiene in order to protect animal health. It contains 2000-10000 ppm ozone.
Hygiene product presented in the form of spray or solution for wound and skin care, disinfection, hygiene for human health. It contains 2000-10000 ppm ozone.
Food solution used in the food industry by dipping or spraying for the preservation of meats, fruits and vegetables, pesticide removal and antibacterial effect. It contains 300-3000 ppm ozone.
Food solution, which is used by dipping or spraying method to prevent spoilage of dried foods and legumes, to provide insecticide and repellent effect. It contains 500-3000 ppm ozone.
Food solution served as a food supplement, as a capsule, as a single-use beverage or in combination with other beverages and sweeteners. It contains 100-1000 ppm ozone.
Disposable or multiple wet wipes. It contains 1000-10000 ppm ozone.
Environment, surface, device cleaner or detergents. It contains 1000-10000 ppm ozone.
A plant solution that provides antibacterial, antifungal, antiparasitic and repellent effects on plants and is presented in intensive doses or in spray form suitable for dilution.co It contains 500-2000 ppm ozone.
It contains antibacterial, antiviral, antifungal, antiparasitic fertilizer solution for use in soilless agriculture. It contains 500-5000 ppm ozone.
Seed solution for use in germination so as to develop seeds, to provide antibacterial effect in seed aquaculture and to remove soil pathogens. It contains 500-5000 ppm ozone
Plant solution that both provides brightness and increases plant health in ornamental plants. It contains 200-1000 ppm ozone.
Fuel additive solution mixed with petroleum products to increase vehicle performance in the chemical industry. It contains 1000-20000 ppm ozone.
Solution created to grow plants in oxygen-free environments. It contains 500-3000 ppm ozone.
Ozonated emulsifier preferably polysorbate and water, thymol, carvacrol and other chemotherapeutic agents and solutions in liposome combined with ozonated oil and nanocarriers and cancer drugs such as paclitaxel combined with intravenous or local injection in cancer treatment. It contains 500-5000 ppm ozone.

An exemplary application of the invention is the mesotherapy products, the contents of which are explained below:

Mesotherapy products for skin repair contain ozonated emulsifier nanoparticle liposomes obtained from ozonated oil, amino acid complex, mineral complex, panthotenic acid; in particular, they contain nanoparticle ozone liposomes, l-arginine, glycine, l-histidine, l-isoleucine, l-leucine, l-lysine, l-methionine, l-phenylalanine, l-proline, l-serine, l-threonine, l-tryptophan, l-tyrosine, l-valine, sodium acetate, sodium glycerophosphate, potassium chloride, magnesium chloride, calcium chloride, panthothenic acid, sodium chloride, distilled water.

Anti-aging mesotherapy products for skin contain ozonated emulsifier, nanoparticle liposomes from ozonated oil, vitamin complex, tranexamic acid, acetyl cysteine; in particular, they contain nanoparticle ozone liposomes, thiamine pyrophosphate, riboflavin, pyridoxine hydrochloride, niacinamide, d panthenol, ascorbic acid, glutathione, tranexamic acid, acetyl cysteine, sodium chloride, distilled water.

Mesotherapy products for hair care contain ozonated emulsifier, nanoparticle liposomes obtained from ozonated oil, vitamin complex, biotin, acetyl cysteine, pantothenic acid; in particular, they contain nanoparticle ozone liposomes, thiamine pyrophosphate, riboflavin, pyridoxine hydrochloride, niacinamide, d panthenol, ascorbic acid, biotin, glutathione, acetyl cysteine, magnesium sulfate, sodium chloride, distilled water

—Barrier Solutions Test Analysis Results—

Liposomal Ozone Nanosolutions Accelerated Aging (Stability) and Antibacterial Efficacy Tests:

Accelerated aging (stability) tests in accordance with ASTM F1980 standard on the inventive liposomal ozone nanosolution samples, sterility tests according to the ISO 11737-2 standard and antibacterial activity tests according to the test method adapted from the CLSI M07 A9 standard were carried out. It was determined according to the test method adapted from the CLSI M07 A9 standard that 1.000 ppm and 1.600 ppm applications of the nanosolution had antibacterial (bactericidal) activity against Staphylococcus aureus (ATCC 25923) bacteria after 2 hours and activity against Escherichia coli (ATCC 25922) bacteria after 1 hour. Accelerated Aging Time (AAT) was calculated as 37 days corresponding to 365 days (1 year) Real Time Aging (RT) with accelerated aging (stability) tests of 1.000 ppm and 1.600 ppm applications of nanosolution according to ASTM F1980 standard. Accelerated Aging Time After 37 days, the solutions were found to be stable. After accelerated aging (stability) tests, no bacterial growth was observed in the solutions in sterility tests of 1.000 ppm and 1.600 ppm applications of nanosolution according to ISO 11737-2 standard. After 365 days accelerated aging (stability) of 1.000 ppm and 1.600 ppm applications of nanosolution according to ASTM F1980 standard, according to the test method adapted from the CLSI M07 A9 standard, it was determined that it has antibacterial (bactericidal) activity against Staphylococcus aureus (ATCC 25923) bacteria after 2 hours and against Escherichia coli (ATCC 25922) bacteria after 1 hour.

The antibacterial activities of 1.000 ppm and 1.600 ppm applications of the nanosolution of the invention were determined according to the method adapted from the CLSI M07 A9 standard test method.

Staphylococcus aureus (ATCC 25923) and Escherichia coli (ATCC 25922) bacterial suspensions were adjusted to 1×105 density and applied to 1.000 ppm and 1.600 ppm solutions. At the end of 2-360 minutes, it was transferred to the culture medium and then it was evaluated whether there was any growth in the media. Test results are given in Chart 1 and 2.

CHART 1
Antibacterial efficacy values against Staphylococcus aureus (ATCC 6538)

| | 2 | 10 | 30 | 1 | 2 | 3 | 6 |
|---|---|---|---|---|---|---|---|
| Name of the | minutes | minutes | minutes | hour | hours | hours | hours |
| sample | Bacteria growth | | | | | | |
| 1.000 ppm | Present | Present | Present | Present | None | None | None |
| 1.600 ppm | Present | Present | Present | Present | None | None | None |

CHART 2
Antibacterial values against Escherichia coli (ATCC 25922)

| | 2 | 10 | 30 | 1 | 2 | 3 | 6 |
|---|---|---|---|---|---|---|---|
| Name of the | minutes | minutes | minutes | hour | hours | hours | hours |
| sample | Bacteria growth | | | | | | |
| 1.000 ppm | Prese | Present | Present | None | None | None | None |
| 1.600 ppm | Prese | Present | Present | None | None | None | None |

It was determined according to the test method adapted from the CLSI M07 A9 standard that 1.000 ppm and 1.600 ppm applications of the nanosolution had antibacterial (bactericidal) activity against Staphylococcus aureus (ATCC 25923) bacteria after 2 hours and activity against Escherichia coli (ATCC 25922) bacteria after 1 hour.

Accelerated aging (stability) tests of 1,000 ppm and 1,600 ppm applications of the inventive liposomal ozone nanosolution were determined according to the ASTM F1980 standard. 1.000 ppm and 1.600 ppm applications of nanosolution were exposed to accelerated aging (stability) in a Nüve FN 120 brand oven set at 55° C. and the accelerated aging (stability) time was calculated with the Arrhenius Equation [Q10(TAA−TRT)/10]. Test results are given in Chart 3.

Chart 3. Accelerated aging (stability) time

| Name of the | Accelerated aging | Accelerated Aging | Aging |
|---|---|---|---|
| sample | temperature (AFF) | Time (AAT) | (Stability) |
| 1.000 ppm | 9.85 | 37.06 | Stable |
| 1.600 ppm | 9.85 | 37.06 | Stable |

Accelerated Aging Time (AAT) was calculated as 37 days corresponding to 365 days (1 year) Real Time Aging (RT) with accelerated aging (stability) tests of 1.000 ppm and 1.600 ppm applications of nanosolution according to ASTM F1980 standard. Accelerated Aging Time After 37 days, the solutions were found to be stable.

After Accelerated aging (stability) tests of the inventive liposomal ozone nanosolution, sterility tests of 1.000 ppm and 1.600 ppm applications of nanosolution were determined according to ISO 11737-2 standard. In 1.000 ppm and 1.600 ppm applications of nanosolution, it was observed whether aerobic mesophilic bacteria grow in Triptone soy broth medium at a temperature of 30±2° C. and for 14 days, whether anaerobic mesophilic bacteria grow in liquid Thioglycolate medium at 30±2° C. and for 14 days, whether bacteria grow in the negative control of Triptone soy broth and Liquid Thioglycolate media at 30±2t and for 14 days, whether Bacillus atropheus grow in the positive control of Triptone soy broth and Liquid Thioglycolate media at 30±2° C. for 14 days. Test results are given in Chart 4.

Chart 4. Sterility values of 1.000 ppm and 1.600 ppm applications
of nanosolution after accelerated aging (stability) tests

| Parameters | 1.000 ppm | 1.600 ppm |
|---|---|---|
| Aerobic mesophilic | no growth | no growth |
| Aerobic mesophilic | no growth | no growth |
| Negative control | no growth | no growth |
| Positive control | growth | growth |
| Bacilius atrupheus | | |

After accelerated aging (stability) tests, no bacterial growth was observed in the solutions in sterility tests of 1.000 ppm and 1.600 ppm applications of nanosolution according to ISO 11737-2 standard.

After 365 days accelerated aging (stability) of 1,000 ppm and 1,600 ppm applications of the inventive liposomal ozone nanosolution according to ASTM F1980 standard, antibacterial activities were determined according to the test method adapted from the CLSI M07 A9 standard. After 365 days accelerated aging (stability) according to ASTM F1980 standard, Staphylococcus aureus (ATCC 25923) and Escherichia coli (ATCC 25922) bacterial suspensions were adjusted to 1×10⁵ density and applied to 1.000 ppm and 1.600 ppm solutions. At the end of 2-360 minutes, it was transferred to the culture medium and then it was evaluated whether there was any growth in the media. Test results are given in Chart 5 and 6.

CHART 5
Antibacterial efficacy values against Staphylococcus aureus
(ATCC 6538)

| | 2 | 10 | 30 | 1 | 2 | 3 | 6 |
|---|---|---|---|---|---|---|---|
| Name of the | minutes | minutes | minutes | hour | hours | hours | hours |
| sample | Bacteria growth | | | | | | |
| 1,000 ppm | Present | Present | Present | Present | None | None | None |
| (365 days | | | | | | | |
| aging) | | | | | | | |
| 1,600 ppm | Present | Present | Present | Present | None | None | None |
| (365 days | | | | | | | |
| aging) | | | | | | | |

CHART 6
Antibacterial values against Escherichia coli (ATCC 25922)

| | 2 | 10 | 30 | 1 | 2 | 3 | 6 |
|---|---|---|---|---|---|---|---|
| Name of the | minutes | minutes | minutes | hour | hours | hours | hours |
| sample | Bacteria growth | | | | | | |
| 1,000 ppm | Present | Present | Present | None | None | None | None |
| (365 days aging) | | | | | | | |
| 1.600 ppm | Present | Present | Present | None | None | None | None |
| (365 days aging) | | | | | | | |

After 365 days accelerated aging (stability) of 1.000 ppm and 1.600 ppm applications of nanosolution according to ASTM F1980 standard, according to the test method adapted from the CLSI M07 A9 standard, it was determined that it has antibacterial (bactericidal) activity against Staphylococcus aureus (ATCC 25923) bacteria after 2 hours and against Escherichia coli (ATCC 25922) bacteria after 1 hour.

The antibacterial activities of 2000 ppm and 3000 ppm applications of the nanosolution of the invention were determined according to the method adapted from the CLSI M07 A9 standard test method. Staphylococcus aureus (ATCC 6538) and Escherichia coli (ATCC 25922) bacterial suspensions were adjusted to 1×10⁵ density and applied to 2000 ppm and 3000 ppm solutions. At the end of 2-360 minutes, it was transferred to the culture medium and then it was evaluated whether there was any growth in the media. Test results are given in Chart 6-14.

CHART 6
Liposomal ozone nanosolution + 1% HYALURONIC ACID
E. COLI 25922 2000 ppm

| 1.Tube | 2.Tube | 3.Tube | 4.Tube | 5.Tube | 6.Tube | 7.Tube |
|---|---|---|---|---|---|---|
| 2000 ppm | 1750 ppm | 1500 ppm | 1250 ppm | 1000 ppm | 750 ppm | 500 ppm |
| 2 min. + | 2 min. + | 2 min. + | 2 min. + | 2 min. + | 2 min. + | 2 min. + |
| 10 min. + | 10 min. + | 10 min. + | 10 min. + | 10 min. + | 10 min. + | 10 min. + |
| 30 min. + | 30 min. + | 30 min. + | 30 min. + | 30 min. + | 30 min. + | 30 min. + |
| 1 hour + | 1 hour + | 1 hour + | 1 hour + | 1 hour + | 1 hour + | 1 hour + |
| 2 hours − | 2 hours − | 2 hours − | 2 hours − | 2 hours − | 2 hours | 2 hours |
| | | | | | 3 colonies | 3 colonies |
| 3 hours − | 3 hours − | 3 hours − | 3 hours − | 3 hours − | 3 hours − | 3 hours − |
| 4 hours − | 4 hours − | 4 hours − | 4 hours − | 4 hours − | 4 hours − | 4 hours − |
| 5 hours − | 5 hours − | 5 hours − | 5 hours − | 5 hours − | 5 hours − | 5 hours − |
| 6 hours − | 6 hours − | 6 hours − | 6 hours − | 6 hours − | 6 hours − | 6 hours − |

Chart 7. Liposomal ozone nanosolution + glycerin Throat Spray E. Coli 25922 3000 ppm

| | | | | | |
|---|---|---|---|---|---|
| 1.Tube | 2.Tube | 3.Tube | 4.Tube | 5.Tube | 6.Tube |
| 3000 ppm | 2500 ppm | 2000 ppm | 1500 ppm | 1000 ppm | 500 ppm |
| 2 min. + | 2 min. + | 2 min. + | 2 min. + | 2 min. + | 2 min. + |
| 10 min. + | 10 min. + | 10 min. + | 10 min. + | 10 min. + | 10 min. + |
| 30 min. | 30 min. | 30 min. | 30 min. + | 30 min. + | 30 min. + |
| REDUCTION | REDUCTION | REDUCTION | | | |
| 1 hour + | 1 hour + | 1 hour | 1 hour | 1 hour + | 1 hour + |
| | | 1.2 colonies | REDUCTION | | |
| 2 hours − | 2 hours − | 2 hours − | 2 hours − | 2 hours − | 2 hours + |
| 3 hours − | 3 hours − | 3 hours − | 3 hours − | 3 hours − | 3 hours |
| | | | | | 1 colony |
| 4 hours − | 4 hours − | 4 hours − | 4 hours − | 4 hours − | 4 hours − |
| 5 hours − | 5 hours − | 5 hours − | 5 hours − | 5 hours − | 5 hours − |
| 6 hours − | 6 hours − | 6 hours − | 6 hours − | 6 hours − | 6 hours − |

CHART
2000 ppm 1-year E. Coli 25922 2000 ppm

| 1.Tube | 2.Tube | 3.Tube | 4.Tube | 5.Tube | 6.Tube | 7.Tube |
|---|---|---|---|---|---|---|
| 2000 ppm | 1750 ppm | 1500 ppm | 1250 ppm | 1000 ppm | 750 ppm | 500 ppm |
| 2 min. | 2 min. + | 2 min. + | 2 min. + | 2 min. + | 2 min. + | 2 min. + |
| REDUCTION | | | | | | |
| 10 min. − | 10 min. | 10 min. | 10 min. | 10 min. + | 10 min. + | 10 min. + |
| | REDUCTION | REDUCTION | REDUCTION | | | |
| 30 min. − | 30 min. − | 30 min. − | 30 min. − | 30 min. | 30 min. + | 30 min. + |
| | | | | REDUCTION | | |
| 1 hour − | 1 hour − | 1 hour − | 1 hour − | 1 hour − | 1 hour − | 1 hour + |
| 2 hours − | 2 hours − | 2 hours − | 2 hours − | 2 hours − | 2 hours | 2 hours |
| | | | | | 3 colonies | 3 colonies |
| 3 hours − | 3 hours − | 3 hours − | 3 hours − | 3 hours − | 3 hours − | 3 hours − |
| 4 hours − | 4 hours − | 4 hours − | 4 hours − | 4 hours − | 4 hours − | 4 hours − |
| 5 hours − | 5 hours − | 5 hours − | 5 hours − | 5 hours − | 5 hours − | 5 hours − |
| 6 hours − | 6 hours − | 6 hours − | 6 hours − | 6 hours − | 6 hours − | 6 hours − |

CHART 9
Ozonated oil and ozonated emulsifier 2000 ppm E. Coli 25922 2000 ppm

| 1st Tube | 2nd Tube | 3rd Tube | 4th Tube | 5th Tube | 6th Tube | 7th Tube |
|---|---|---|---|---|---|---|
| 2000 ppm | 1750 ppm | 1500 ppm | 1250 ppm | 1000 ppm | 750 ppm | 500 ppm |
| 2 min. + | 2 min. + | 2 min. + | 2 min. + | 2 min. + | 2 min. + | 2 min. + |
| 10 min. + | 10 min. + | 10 min. + | 10 min. + | 10 min. + | 10 min. + | 10 min. + |
| 30 min. + | 30 min. + | 30 min. + | 30 min. + | 30 min. + | 30 min. + | 30 min. + |
| 1 hour + | 1 hour + | 1 hour + | 1 hour + | 1 hour + | 1 hour + | 1 hour + |
| 2 hours − | 2 hours | 2 hours | 2 hours | 2 hours | 2 hours + | 2 hours + |
| | REDUCTION | REDUCTION | REDUCTION | REDUCTION | | |
| 3 hours − | 3 hours − | 3 hours − | 3 hours | 3 hours | 3 hours | 3 hours |
| | | | 2 colonies | REDUCTION | REDUCTION | REDUCTION |
| 4 hours − | 4 hours − | 4 hours − | 4 hours − | 4 hours − | 4 hours − | 4 hours |
| | | | | | | REDUCTION |
| 5 hours − | 5 hours − | 5 hours − | 5 hours − | 5 hours − | 5 hours − | 5 hours |
| | | | | | | 2 colonies |
| 6 hours − | 6 hours − | 6 hours − | 6 hours − | 6 hours − | 6 hours − | 6 hours − |

CHART 10
Liposomal ozone nanosolution 2000 ppm + 0.5% Thymol E. Coli 25922 2000 ppm

| 1st Tube | 2nd Tube | 3rd Tube | 4th Tube | 5th Tube | 6th Tube | 7th Tube |
|---|---|---|---|---|---|---|
| 2000 ppm | 1750 ppm | 1500 ppm | 1250 ppm | 1000 ppm | 750 ppm | 500 ppm |
| 2 min. + | 2 min. + | 2 min. + | 2 min. + | 2 min. + | 2 min. + | 2 min. + |
| 10 min. − | 10 min. − | 10 min. − | 10 min. − | 10 min. − | 10 min. | 10 min. |
| | | | | | 5 colonies | REDUCTION |
| 30 min. − | 30 min. − | 30 min. − | 30 min. − | 30 min. − | 30 min. − | 30 min. − |
| 1 hour − | 1 hour − | 1 hour − | 1 hour − | 1 hour − | 1 hour − | 1 hour − |
| 2 hours − | 2 hours − | 2 hours − | 2 hours − | 2 hours − | 2 hours − | 2 hours − |
| 3 hours − | 3 hours − | 3 hours − | 3 hours − | 3 hours − | 3 hours − | 3 hours − |
| 4 hours − | 4 hours − | 4 hours − | 4 hours − | 4 hours − | 4 hours − | 4 hours − |
| 5 hours − | 5 hours − | 5 hours − | 5 hours − | 5 hours − | 5 hours − | 5 hours − |
| 6 hours − | 6 hours − | 6 hours − | 6 hours − | 6 hours − | 6 hours − | 6 hours − |

CHART 11
Ozonated oil + ozonated
emulsifier 2000 ppm S.auneus 6538 2000 ppm

| 1st | 2nd | 3rd | 4th | 5th | 6th | 7th |
|---|---|---|---|---|---|---|
| Tube | Tube | Tube | Tube | Tube | Tube | Tube |
| 2000 | 1750 | 1500 | 1250 | 1000 | 750 | 500 |
| ppm | ppm | ppm | ppm | ppm | ppm | ppm |
| 2 min. | 2 min. | 2 min. | 2 min. | 2 min. | 2 min. | 2 min. |
| + | + | + | + | + | + | + |
| 10 min. | 10 min. | 10 min. | 10 min. | 10 min. | 10 min. | 10 min. |
| + | + | + | + | + | + | + |
| 30 min. | 30 min. | 30 min. | 30 min. | 30 min. | 30 min. | 30 min. |
| + | + | + | + | + | + | + |
| 1 hour | 1 hour | 1 hour | 1 hour | 1 hour | 1 hour | 1 hour |
| + | + | + | + | + | + | + |
| 2 hours | 2 hours | 2 hours | 2 hours | 2 hours | 2 hours | 2 hours |
| + | + | + | + | + | + | + |
| 3 hours | 3 hours | 3 hours | 3 hours | 3 hours | 3 hours | 3 hours |
| + | + | + | + | + | + | + |
| 4 hours | 4 hours | 4 hours | 4 hours | 4 hours | 4 hours | 4 hours |
| − | − | − | − | − | + | + |
| 5 hours | 5 hours | 5 hours | 5 hours | 5 hours | 5 hours | 5 hours |
| − | − | − | − | − | + | + |
| 6 hours | 6 hours | 6 hours | 6 hours | 6 hours | 6 hours | 6 hours |
| − | − | − | − | − | − | − |

CHART 12
Liposomal ozone nanosolution
2000 ppm + 0.5% Thymol S.auneus 6538 2000 ppm

| 1st | 2nd | 3rd | 4th | 5th | 6th | 7th |
|---|---|---|---|---|---|---|
| Tube | Tube | Tube | Tube | Tube | Tube | Tube |
| 2000 | 1750 | 1500 | 1250 | 1000 | 750 | 500 |
| ppm | ppm | ppm | ppm | ppm | ppm | ppm |
| 2 min. | 2 min. | 2 min. | 2 min. | 2 min. | 2 min. | 2 min. |
| + | + | + | + | + | + | + |
| 10 min. | 10 min. | 10 min. | 10 min. | 10 min. | 10 min. | 10 min. |
| REDUCTION | − | − | + | + | + | + |
| 30 min. | 30 min. | 30 min. | 30 min. | 30 min. | 30 min. | 30 min. |
| + | − | − | − | + | + | + |
| 1 hour | 1 hour | 1 hour | 1 hour | 1 hour | 1 hour | 1 hour |
| REDUCTION | − | − | REDUCTION | + | + | + |
| 2 hours | 2 hours | 2 hours | 2 hours | 2 hours | 2 hours | 2 hours |
| REDUCTION | − | − | − | − | + | + |
| 3 hours | 3 hours | 3 hours | 3 hours | 3 hours | 3 hours | 3 hours |
| − | − | − | − | − | − | REDUCTION |
| 4 hours | 4 hours | 4 hours | 4 hours | 4 hours | 4 hours | 4 hours |
| − | − | − | − | − | − | − |
| 5 hours | 5 hours | 5 hours | 5 hours | 5 hours | 5 hours | 5 hours |
| − | − | − | − | − | − | − |
| 6 hours | 6 hours | 6 hours | 6 hours | 6 hours | 6 hours | 6 hours |
| − | − | − | − | − | − | − |

CHART 13
Liposomal ozone nanosolution + glycerin Throat Spray E.Coli 3000 ppm

| 1st | 2nd | 3rd | 4th | 5th | 6th | 7th |
|---|---|---|---|---|---|---|
| Tube | Tube | Tube | Tube | Tube | Tube | Tube |
| 2 min. | 2 min. | 2 min. | 2 min. | 2 min. | 2 min. | 2 min. |
| + | + | + | + | + | + | + |
| 10 min. | 10 min. | 10 min. | 10 min. | 10 min. | 10 min. | 10 min. |
| + | + | + | + | + | + | + |
| 30 min. | 30 min. | 30 min. | 30 min. | 30 min. | 30 min. | 30 min. |
| + | + | + | + | + | + | + |
| 1 hour | 1 hour | 1 hour | 1 hour | 1 hour | 1 hour | 1 hour |
| − | − | − | − | − | − | + |
| 2 hours | 2 hours | 2 hours | 2 hours | 2 hours | 2 hours | 2 hours |
| 3 hours | 3 hours | 3 hours | 3 hours | 3 hours | 3 hours | 3 hours |
| 4 hours | 4 hours | 4 hours | 4 hours | 4 hours | 4 hours | 4 hours |
| 5 hours | 5 hours | 5 hours | 5 hours | 5 hours | 5 hours | 5 hours |
| 6 hours | 6 hours | 6 hours | 6 hours | 6 hours | 6 hours | 6 hours |
| − | − | − | − | − | − | − |

CHART 14
Liposomal ozone nanosolution 2000 ppm 1 year S.auneus 6538 2000 ppm

| 1st | 2nd | 3rd | 4th | 5th | 6th | 7th |
|---|---|---|---|---|---|---|
| Tube | Tube | Tube | Tube | Tube | Tube | Tube |
| 2000 | 1750 | 1500 | 1250 | 1000 | 750 | 500 |
| ppm | ppm | ppm | ppm | ppm | ppm | ppm |
| 2 min. | 2 min. | 2 min. | 2 min. | 2 min. | 2 min. | 2 min. |
| + | + | + | + | + | + | + |
| 10 min. | 10 min. | 10 min. | 10 min. | 10 min. | 10 min. | 10 min. |
| + | + | + | + | | + | + |
| 30 min. | 30 min. | 30 min. | 30 min. | 30 min. | 30 min. | 30 min. |
| + | + | + | + | + | + | + |
| 1 hour | 1 hour | 1 hour | 1 hour | 1 hour | 1 hour | 1 hour |
| − | − | REDUCTION | REDUCTION | REDUCTION | + | + |
| | | (−) | (+) | (−) | | |
| 2 hours | 2 hours | 2 hours | 2 hours | 2 hours | 2 hours | 2 hours |
| REDUCTION | − | − | 4 colonies | − | + | + |
| 3 hours | 3 hours | 3 hours | 3 hours | 3 hours | 3 hours | 3 hours |
| − | − | REDUCTION | 2 colonies | − | − | 5 colonies |
| 4 hours | 4 hours | 4 hours | 4 hours | 4 hours | 4 hours | 4 hours |
| − | − | − | − | − | − | 2 colonies |
| 5 hours | 5 hours | 5 hours | 5 hours | 5 hours | 5 hours | 5 hours |
| − | − | − | − | − | − | − |
| 6 hours | 6 hours | 6 hours | 6 hours | 6 hours | 6 hours | 6 hours |
| − | − | − | − | − | − | − |

After 365 days accelerated aging (stability) of 2000 ppm and 3000 ppm applications of nanosolution according to ASTM F1980 standard, according to the test method adapted from the CLSI M07 A9 standard, it has been determined that it has antibacterial (bactericidal) activity at an increasing rate against Staphylococcus aureus bacteria and Escherichia coli bacteria.

Skin Irritation Test of Liposomal Ozone Nanosolutions:

The inventive irritation test of liposomal ozone nanosolution was carried out considering TS EN ISO 10993-10:2010 standard, the experimental animal used in the test is considered according to TS EN ISO 10993-2:2006 standard, the material preparation is made according to TS EN ISO 10993-12:2013 standard guidelines. Three healthy young New Zealand albino rabbits with a body weight of 2-3 kg were used as experimental animals.

Test material given under the title of TS EN ISO 10993-10:2010 annex A A.2,2 Liquid test materials; Liquids were applied without dilution or by direct precipitation or, if not applicable, at a dose of 1600 ppm, which is the usage dose according to the guideline. Sodium lauryl sulphate (SLS) was used as a positive control and distilled water was used as a negative control. The sponge impregnated with the nanosolution of the invention was kept in direct contact with the sample application areas. SLS impregnated sponge was applied to the positive control area. The samples were covered with gauze, fixed with a bandage, and topically contacted with the back skin for 4 hours. The primary irritation index was determined by evaluating the application areas (1±0.1) s, (24±2) s, (48±2) s, and (72±2) hours after the 4th hour application, according to the skin reaction score given in Chart 1. As stated in the standard, the 1st hour was not included in the calculation.

Chart 1. Scoring system for skin reaction

| | Irritation |
|---|---|
| Reaction | score |
| Erythema and Eschar Formation | |
| No erythema | 0 |
| Very mild erythema (difficult to detect) | 1 |
| Well-defined erythema | 2 |
| Moderate erythema | 3 |
| Between severe erythema (beet-like red) and eschar | 4 |
| formation, which prevents grading of the erythema | |
| Edema formation: | |
| No edema | 0 |
| Very mild edema | 1 |
| Significant edema (Marked edema area borders dyspepsy) | 2 |
| Moderate edema (approximately 1 mm increased) | 3 |
| Severe edema (increased more than 1 mm and | 4 |
| protruded beyond the | |
| Highest possible score for irritation | 8 |

Chart 2. Evaluation Chart for Primary or
Cumulative Irritation Index categories

| Average score | Response Classification |
|---|---|
| 0-0.4 | Insignificant |
| 0.5-1.9 | Mild |
| 2-4.9 | Moderate |
| 5-8 | Severe |

The observations made according to the TS EN ISO 10993-10:2010 standard and their evaluations are given in Chart 3 and Chart 4.

CHART 3
Irritation score

| | | | Observation | | | | | | | |
|---|---|---|---|---|---|---|---|---|---|---|
| | | | Erythem | | | | Edema | | | |
| Animal | | Application | | 24th | 48th | 72 | | 24th | 43rd | 72 |
| No | Groups | areas | hour | hour | hour | nd | hour | hour | hour | nd |
| 1 | Sample | Left front | 1 | 0 | 0 | 0 | 0 | 0 | 0 | 0 |
| | | Right back | 1 | 0 | 0 | 0 | 0 | 0 | 0 | 0 |
| | Positive | Right front | 4 | 4 | 3 | 1 | 3 | 2 | 2 | 1 |
| | control | | | | | | | | | |
| | Negative | Left back | 0 | 0 | 0 | 0 | 0 | 0 | 0 | 0 |
| | control | | | | | | | | | |
| 2 | Sample | Left front | 1 | 1 | 0 | 0 | 0 | 0 | 0 | 0 |
| | | Right back | 0 | 0 | 0 | 0 | 0 | 0 | 0 | 0 |
| | Positive | Right front | 3 | 3 | 2 | 1 | 3 | 2 | 0 | 0 |
| | control | | | | | | | | | |
| | Negative | Left back | 0 | 0 | 0 | 0 | 0 | 0 | 0 | 0 |
| | control | | | | | | | | | |
| 3 | Sample | Left front | 0 | 0 | 0 | 0 | 0 | 0 | 0 | 0 |
| | | Right back | 0 | 0 | 0 | 0 | 0 | 0 | 0 | 0 |
| | Positive | Right front | 3 | 3 | 2 | 1 | 2 | 2 | 1 | 0 |
| | control | | | | | | | | | |
| | Negative | Left back | 0 | 0 | 0 | 0 | 0 | 0 | 0 | 0 |
| | control | | | | | | | | | |

Chart 4. Score average

| | Primary Irritation | | | Primary |
|---|---|---|---|---|
| | Score | | | Index |
| Example | Rabbit 1 | Rabbit 2 | Rabbit 3 | Irritation |
| Sample | 0.0 + 0.0 | 0.083 + 0.2 | 0.0 + 0.0 | 0.027 |
| Positive control | 2.167 + 1.169 | 1.333 + 1.2 | 1.500 + 1.049 | 1.666 |
| Negative control | 0.0 + 0.0 | 00 + 0.0 | 00 + 0.0 | 0.0 |

Irritation Test result in TS EN ISO 10993-10:2010 Standards

In the evaluation made based on the observation values (Chart 3, 4) made at the 24th, 48th, and 72nd hours after the application in the irritation test of the inventive nanosolution, it was determined that the samples did not cause significant erythema and edema in any of the subjects. It has been determined according to the test results carried out in line with the directive of the TS EN ISO 10993-10:2010 standard that the inventive nanosolution does not have any irritating effect.

In Vitro Cytotoxicity Test of Liposomal Ozone Nanosolutions:

Cytotoxicity refers to the rate of toxic effects on living cells. Cytotoxicity tests are tests that are evaluated by considering the cell proliferation rate and the toxic effect on the cell in the appropriate cell culture of the substance considered to be toxic. These test systems are carried out for morphological observation of cellular damage, determination of cellular damage by various measurement methods, determination of cellular growth, determination of any change in cellular metabolism. Cytotoxicity tests can be performed in vivo or in vitro. In in vitro tests, the substance whose cytotoxicity is investigated is administered to cells in increasing concentrations. The effects of this substance on cell morphology and cell survival rates are investigated.

MTT method [3-(4,5-dimethylthiazol-2-yl)-2,5-diphenyltetrazolium bromide]: In this method, MTT is reduced to formazan, while the color formed is measured colorimetrically. The amount of formazan produced gives the number of viable cells. MTT is a substance that is actively absorbed into cells and reduced to colored, water-insoluble formazan by a mitochondria-dependent reaction. The MTT reduction property of the cells is taken as a measure of cell viability and the dye density obtained as a result of MTT analysis correlates with the number of viable cells.

The test system used in this research is based on the following standards;

TS EN ISO 10993-5:2009, Biological evaluation of medical devices—Part 5: Tests for in vitro cytotoxicity. TS EN ISO 10993-1:2018, Biological evaluation of medical devices—Part 1: Evaluation and testing. TS EN ISO 10993-5:2009, Biological evaluation of medical devices—Part 12: Sample preparation and reference materials. USP 31, 2008, Chapter 87—Biological reactivity tests, in vitro.

Extraction of test material, negative and positive groups was carried out with 1600 ppm liposomal ozone nanosolution at 37° C. for 24 hours in accordance with TS EN ISO 10993-12:2012 standards.

The BALB/c 3T3 (CCL-163) cell line from ATCC was used for the cytotoxicity test study. Cells were propagated in DMEM (dulbecco's modified eagle medium) (ATCC Cat No: 30-2006) medium supplemented with 10% FBS (fetal bovine serum) and 2% glutamine and incubated at 37° C. in an oven with 5% CO2. A mixture of 0.25% trypsin and 0.03% EDTA was used for trypsinase of cells as recommended by ATCC. Cells were suspended in culture medium and 100 μl was transferred to 96-well plates with 104 cells in each well.

After 24 hours of cell culture conditions, the medium on the culture was removed and 100 μl of test material was added from the positive and negative control extracts. All doses were administered with at least 5 repetitions. At the end of 48 hours of waiting in an oven with 5% CO2 at 37° C. and 95% humidity, the culture medium was removed after the plates were examined.

Negative control (NC): Polyethylene tube

Positive Control (PK): Serial dilutions of DMSO (Dimethylsulfoxide) (10-30 v/v)

Test Material (TM) concentrations: 100-30-10-3-v/v

After examining the plates, the culture medium was removed from the wells. 50 μl of MTT solution was added to each test well. The plates were incubated for 2 hours at 37° C. Then, the MTT solution was removed from the wells and 100 μl of isopropanol was added to each well. Absorbance measurements were taken and evaluated with a microplate reader containing a 570 nm filter.

If the viability of the test material is less than 70%, it is considered cytotoxic.

CHART 1
Results of quantitative measurement of cytotoxic effects by MTT Test.

| | Optical density (OD) mean value | | | | | | | |
|---|---|---|---|---|---|---|---|---|
| | 570 nm ± standard deviation (SD) | | | | % Dilution | | | |
| | % Dilution concentrations (v/v) | | | | concentrations (v/v) | | | |
| | 100 | 30 | 10 | 3 | 100 | 30 | 10 | 3 |
| TM | 0.244 ± 0.08 | 0.276 ± 0.05 | 0.29 ± 0.03 | 0.31 ± 0.06 | 76.25 | 86.25 | 90.62 | 96.87 |
| NC | 0.32 ± 0.05 | | | | 100 | | | |
| PC | | 0.078 ± 0.02 | 0.1 ± 0.06 | | | | 24.37 | 31.25 |

| | |
|---|---|
| BLANK | 0.046 ± 0.002 |

The % vitality value was determined according to the formula below.
Vitality % = 100 × OD570TM/OD570_{NK}
OD_{570TM} = It is the average value of the optical density value of the test material after the blank is removed.
OD_{570NK} = It is the average value of the optical density value of the negative control after removing the blank.
According to the test results carried out in line with the directive of the TS EN ISO 10993-5:2009 standard, it was determined that the test material, the liposomal ozone nanosolution of the invention, did not have a cytotoxic effect.

Liposomal Ozone Nanosolutions In Vivo Acute Ocular Irritation Test:

The experiment aims to test the material or product that has the potential to cause ocular irritation. The ocular irritation test is only performed for materials that will come into contact with the eye or eyelid and when safety information cannot be obtained by other means. The test material is carried out in accordance with the directions specified in TS EN ISO 10993-10:2010 annex A. If the material to be tested is liquid, 0.1 ml undiluted (1600 ppm dose) is dripped into the lower conjunctival sac of one eye. If the test material is in the spray pump, it is removed from the pump and tested by dropping 0.1 mL as in liquids. We used 3 healthy young adult albino rabbits of both sexes weighing 2 kg to 3 kg from a single strain. The animals were kept in the environmental conditions specified in TS EN ISO 10993-2:2006. Both eyes of each rabbit are visually inspected up to 24 h prior to the start of the experiment so as to determine if an ocular abnormality is present. When the eyes are examined, 2% sodium fluorescein BP (British Pharmacopoeia) can be used to visualize any corneal damage.

After the test sample was instilled into the conjunctival sac, the eyelids were held together for about 1 hour. For Observation of Animals and determination of the Irritation index, it is examined at approximately (1±0.1) s, (24±2) s, (48±2) s, and (72±2) s. Since no lesion was observed, it was not necessary to advance the observations to longer periods. Observations were graded according to the grading scale of the ocular lesions given in Chart 1.

Chart 1. Grading system of ocular lesions

| | Numerical |
|---|---|
| Reaction | Rating |
| 1-CORNEA | |
| Degree of opacity (the most intensive area) | |
| No opacity | 0 |
| Scattered or diffused areas, details of the iris clearly visible | 1* |
| Easily distinguishable translucent areas, iris details slightly | 2* |
| blurred | |
| Opaque areas, iris details not visible, pupil size barely | 3* |
| distinguishable) | |
| Opaque, details of the iris cannot be seen | 4* |
| Affected corneal area | |
| One quarter area (or less), not zero | 0 |
| More than a quarter of the area, less than half | 1 |
| Wider than half: but less than three-quarters | 2 |
| More than three-quarters wide up to the whole area | 3 |
| 2-IRIS | |
| Normal | 0 |
| Abnormal folds, congestion swelling, pericorneal injection | 1* |
| (any or all, or a combination thereof), iris still | |
| responding to light (lazy reaction positive) | |
| Unresponsive to light, bleeding, major destruction | 2* |
| (any or all) | |
| 3. Conjunctivae | |
| Redness [pleural (of the eyelid) and bulbar (under the eye) | |
| conjunctiva, excluding cornea and iris] | |
| Veins are normal | 0 |
| The veins are completely filled above normal | 1* |
| More prevalent, deep red (deeper crimson red), each vein | 2* |
| not easily | |
| Prevalent dark red (beefy red) | 3* |
| Chemosis | |
| No swelling | 0 |
| Abnormal swelling (including nictitan membrane) | 1* |
| Significant swelling with partial outward turning of the | 2* |
| eyelids | |
| Swelling with half-closed eyelids | 3* |
| Swelling with approximately half-closed to fully closed | 4* |
| eyelid | |
| Discharge | |
| No discharge | 0 |
| Any amount different from normal (except for small | 1 |
| amounts observed in the inner canthus of normal animals) | |
| Discharge with moistening of the eyelids and adjoining | 2 |
| hairs | |
| Discharge and significant area around the eyes with | 2 |
| moistening of the Positive result | |

0.1 ml of the product of the nanosolution of the invention was instilled In the left eye of the rabbits, both eyes were examined and evaluated approximately (1±0.1) hour after instillation. No indication of any conjunctival irritation caused by the application was observed. Since no signs of permanent or other corneal irritation were observed in the examination performed by instilling 2% sodium fluorescein, it was not necessary to extend the observation period. Evaluations were graded according to Table 1. No positive reaction was observed in any animal. In animals, no ocular changes such as mild or conjunctival membrane peeling and ulceration, corneal perforation (perforation), blood or pus in the anterior chamber of the eye) or—Bloody or hairy (purulent) discharge, or—Severe corneal ulceration were found.

CHART 2
Grading system of ocular lesions

| | Numerical |
|---|---|
| Reaction | Rating |
| 1-Cornea | 0 |
| 2-lris | 0 |
| 3. Conjunctivae | |
| Redness [pleural (of the eyelid) and bulbar (under the eye) | 0 |
| Chemosis | 0 |
| Discharge | 0 |

When the eyes treated with control eyes in the ocular irritation test of the liposomal ozone nanosolution of the invention are evaluated, it was determined that there was no difference, and that the nanosolution of the invention did not cause any changes reflecting any irritation in ocular structures (Conjunctival, cornea, iris). It has been determined according to the observations of the “Ocular Irritation Test” conducted in line with the directive of the TS EN ISO 10993-10:2010 standard that the inventive liposomal ozone nanosolution has no ocular irritation effect.

Oral Mucosal Irritation Test of Liposomal Ozone Nanosolutions:

Oral mucosal irritation test was carried out considering the TS EN ISO 10993-10:2010 standard, the experimental animal used in the test is considered according to TS EN ISO 10993-2:2006 standard, the material preparation is made according to TS EN ISO 10993-12:2012 standard guidelines. The test is performed for materials intended to come into contact with oral tissue and where safety data cannot be obtained by other means.

Animals were acclimated to the environment by taking care of them as specified in TS EN ISO 10993-2:2006. Healthy young adult Syrian hamsters of both sexes, unrelated to a single strain, were used for testing. Product sample-impregnated gauze was placed in the inner cheek pouch of each animal under ketamine/Xylazine anesthesia. The exposure time was evaluated according to the rule of reflecting the actual expected use time of the material, and the sample was kept in the inner cheek pouch for 1 hour.

Test material given under the title of ISO 10993-10:2010 annex A A.2,2 Liquid test materials; Liquids were applied without dilution or by direct precipitation or, if not applicable, at a dose of 1600 ppm, which is the usage dose according to the guideline. Saline solution-impregnated absorbent gauze was used as negative control. As a positive control, HCl acid solution adjusted to pH 1.5 was used in our laboratory based on the rule that liquids with pH 2 and below are considered irritating.

The left inner cheek pouch of each animal was evaluated for the experimental sample, and the contralateral (Right) inner cheek was considered as the negative control. No material was placed into the right side inner cheek pouch, it was only washed with physiological saline and dissected at the end of the experiment.

Gauze cloth impregnated with a usage dose of 1600 ppm of the nanosolution of the invention was placed in the left inner cheek pouch of Syrian hamsters under ketamine/Xylazine anesthesia, whose inner cheeks were emptied and washed with saline. After the exposure of the subject to the sample for 1 hour, the sample was removed and the inner cheek was rinsed with saline solution, taking care not to contaminate the other cheek. Experiment (left) and control (right) inner cheek pouches were evaluated macroscopically according to chart 1. The experiment was terminated after 1 hour of contact.

CHART 1
Rating system for Oral and Penile reactions

| | Numerical |
|---|---|
| Reaction | Rating |
| Erythema and eschar formation | |
| No erythema | 0 |
| Very mild erythema (difficult to detect) | 1 |
| Evident erythema | 2 |
| Moderate erythema | 3 |
| Between severe erythema (beet-like red) and eschar | 4 |
| formation that prevents grading of the erythema | |

Other adverse changes in tissues should be recorded and reported.

According to the standard directive, the exposure time should reflect the actual expected use time of the material, but should not be less than 5 minutes. The repeated observation rating (Table 1) performed at long-term chronic exposures is summed up and divided by the total repeat application 4. Since it is a device with subacute use, the application was performed 4 times with one hour intervals. Observations of the sample applied left inner cheek were compared with the right inner cheeks (negative control) washed with saline.

CHART 2
Grading system for microscopic examination of oral, penile, rectal and
vaginal tissue reaction

| Reaction | Numerical Rating |
|---|---|
| Epithelium | |
| Normal, durable | 0 |
| Cell degeneration or flattening | 1 |
| Metaplasia | 2 |
| Focal erosion | 3 |
| General erosion | 4 |
| Leukocyte infiltration (for each high | |
| penetration area) | |
| None | 0 |
| At least (less than 25) | 1 |
| Mild (26 to 50) | 2 |
| Moderate (51 to 100) | 3 |
| Prominent (greater than 100) | 4 |
| Vascular congestion | |
| None | 0 |
| At least | 1 |
| Mild | 2 |
| Moderate | 3 |
| Prominent with the destruction of the veins | 4 |
| Edema | |
| None | 0 |
| At least | 1 |
| Mild | 2 |
| Moderate | 3 |
| Prominent | 4 |

CHART 3
Irritation Index
Irritation Index

| | |
|---|---|
| 0 | No |
| 1-4 | At least |
| 5-8 | Mild |
| 9-11 | Moderate |
| 12-16 | Severe |

The observations made according to the TS EN ISO 10993-10:2010 standard and their evaluations are given in Chart-4 3. In the macroscopic examination, no irritation appearance (erythema or eschar) was observed in the sample-applied inner cheek mucosa, similar to the controls. Areas of mucosal erosion were noted in the positive controls. The mean irritation index was calculated as zero (0) for each animal except the positive control.

CHART 3
Macroscopic irritation score

| | | Observation | |
|---|---|---|---|
| Animal | Application areas | Erythema | Eschar |
| 1 | Left cheek | 0 | 0 |
| | Right cheek (Negative | 0 | 0 |
| 2 | Left cheek | 0 | 0 |
| | Right Cheek | 0 | 0 |
| 3 | Left cheek | 0 | 0 |
| | Right Cheek | 0 | 0 |
| Positive | Left Cheek (Positive | 4 | 3 |
| Control | Right Cheek | 0 | 0 |

CHART 4
Microscopic irritation score

| | Numerical Rating | | | | | | | |
|---|---|---|---|---|---|---|---|---|
| | 1. Subject | | 2. Subject | | 3. Subject | | Positive Control | |
| | Left | Cheek | Left | Right | Left | Right | Left | Right |
| | Cheek | (Negative | Cheek | Cheek | Cheek | Cheek | Cheek | Cheek |
| Reaction | (Experiment) | Control) | (Experiment) | Negative | (Experiment) | (Negative | (Experiment) | (Negative |
| Epithelium | 0 | 0 | 0 | 0 | 0 | 0 | 3 | 0 |
| Leukocyte | 0 | 0 | 0 | 0 | 0 | 0 | 2 | 0 |
| infiltration | | | | | | | | |
| (for each high | | | | | | | | |
| penetration | | | | | | | | |
| Vascular | 0 | 0 | 0 | 0 | 0 | 0 | 1 | 0 |
| conoestio | | | | | | | | |
| Edema | 0 | 0 | 0 | 0 | 0 | 0 | 0 | 0 |

When the Hematoxylin-Eosin (HE) stained preparations prepared from the inner cheek samples taken for histopathological examination from the test product applied group are examined under the microscope, there was no erosion or ulceration in the mucosa, epithelium, leukocyte infiltration and vascular congestion.

After Hematoxylin-Eosin (HE) stained preparations prepared from inner cheek samples taken for histopathological examination were examined under a microscope, it was evaluated whether there was any erosion or ulceration in the epithelium in the mucosa, leukocyte infiltration and vascular congestion. No findings reflecting the irritating appearance of the oral mucosal structures of the application of the product samples of the nanosolution of the invention were found in the subjects in the light microscopic histopathological examination. It was determined that the nanosolution of the invention did not have an irritating effect on the oral mucosa according to the results of the macroscopic and histopathological evaluations of the oral mucosal irritation test applied for materials likely to come into contact with the oral tissue (TS EN ISO 10993-10).

Mucous Irritation Test of Liposomal Ozone Nanosolutions:

Nasal mucosal irritation test was carried out considering the TS EN ISO 10993-10:2010 standard, the experimental animal used in the test is considered according to TS EN ISO 10993-2:2006 standard, the material preparation is made according to TS EN ISO 10993-12:2012 standard guidelines. The test is performed for materials intended to come into contact with nasal tissue and where safety data cannot be obtained by other means.

Animals were acclimated to the environment by taking care of them as specified in TS EN ISO 10993-2:2006. Twelve healthy young adult BalbC mice of both sexes (6 experiments, 6 controls) unrelated to a single strain were used for testing. A single dose (1 ml) of 1600 ppm nanosolution of the invention was instilled into the nasal cavity of each animal for 4 days.

Test material given under the title of ISO 10993-10:2010 annex A A.2,2 Liquid test materials; Liquids were applied without dilution or by direct precipitation or, if not applicable, by diluting with a suitable liquid at a dose of 1600 ppm, which is the usage dose according to the guideline. The comparison was made with the Negative control group using only saline. Positive control 4-day irritant application was not applied because it is not ethical in terms of animal welfare.

The experimental group was formed by dripping a single dose (1 ml) of 1600 ppm nanosolution of the invention into the nasal cavity for 4 days. The control group was formed by dripping the same amount of saline nanosolution into the nasal cavity for 4 days.

After 4 days of application, the noses of the subjects who were euthanized under high-dose Ketamine/Xylazine anesthesia were dissected and taken into decalcification process to soften the bones after 10% formalin fixative.

CHART 1
Rating system for nasal reactions

| | Numerical |
|---|---|
| Reaction | Rating |
| Erythema and eschar formation | |
| No Erythema | 0 |
| Very mild erythema (difficult to detect) | 1 |
| Evident erythema | 2 |
| Moderate erythema | 3 |
| Between severe erythema (beet-like red) and eschar | 4 |
| formation, which prevents grading of the erythema | |

Other adverse changes in tissues should be recorded and reported.

The animals were observed clinically during the 4-day administration of the test sample. No signs of nasal redness, discharge or restlessness were observed in the experimental and control group subjects. The animals displayed normal feeding and social behavior.

Microscopic evaluation in the nasal mucosal irritation test was performed by considering the parameters examined in the vaginal, rectal and penile irritation tests (Chart 2).

CHART 2
Grading system for microscopic examination of oral, penile, rectal
and vaginal tissue reaction

| Reaction | Numerical Rating |
|---|---|
| Epithelium | |
| Normal, intact | 0 |
| Cell degeneration or flattening | 1 |
| Metaplasia | 2 |
| Focal erosion | 3 |
| General erosion | 4 |
| Leukocyte infiltration (for each high | |
| None | 0 |
| At least (less than 25) | 1 |
| Mild (between 26 to 50) | 2 |
| Moderate (51 to 100) | 3 |
| Prominent (more than 100) | 4 |
| Vascular congestion | |
| None | 0 |
| At least | 1 |
| Mild | 2 |
| Moderate | 3 |
| Prominent with the destruction of the veins | 4 |
| Edema | |
| None | 0 |
| At least | 1 |
| Mild | 2 |
| Moderate | 3 |
| Prominent | 4 |

CHART 3
Irritation Index
Irritation Index

| | |
|---|---|
| 0 | No |
| 1-4 | At least |
| 5-8 | Mild |
| 9-11 | Moderate |
| 12-16 | Severe |

Macroscopic observations and microscopic evaluations made according to TS EN ISO 10993-10:2010 standard are given in Chart 1. In the macroscopic examination, no irritation appearance (erythema or eschar) was observed in the sample-applied nasal mucosa, similar to the controls. Areas of mucosal erosion were noted in positive controls. The mean irritation index was calculated as zero (0) for each animal except the positive control.

CHART 1
Microscopic irritation score

| | | | Leukocyte | | | |
|---|---|---|---|---|---|---|
| | | | infiltration | | | |
| | | | (for each | | | |
| | Animal | | high | Vascular | | Irritation |
| Groups | No | Epithelium | penetration | congestion | Edema | Index |
| Neqative | 1 | 0 | 0 | 0 | 0 | 0 |
| Control | 2 | 0 | 0 | 0 | 0 | 0 |
| Group | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | | 0 | 0 | 0 |
| Experiment | 1 | 0 | 0 | 0 | 0 | 0 |
| Group | 2 | 0 | 0 | 0 | 0 | 0 |
| | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | 0 | 0 | 0 | 0 |

After Hematoxylin-Eosin (HE) stained preparations prepared from nasal samples taken for histopathological examination were examined under a microscope, it was evaluated whether there was any erosion or ulceration in the epithelium in the mucosa, leukocyte infiltration and vascular congestion. No findings reflecting the irritating effect of the nasal mucosal structures of the application of the product samples of the nanosolution of the invention were found in the subjects in the light microscopic histopathological examination. It was determined that the nanosolution of the invention did not have an irritating effect on the nasal mucosa according to the results of the macroscopic and histopathological evaluations of the nasal mucosal irritation test applied for materials likely to come into contact with the nasal tissue (TS EN ISO 10993-10).

Sensitization Test of Liposomal Ozone Nanosolutions:

Sensitization test was carried out considering the TS EN ISO 10993-10:2010 standard by the Closed Patch-Buehler Test method, the experimental animal used in the test is considered according to TS EN ISO 10993-2:2006 standard, the material preparation is made according to Annex A standard guidelines.

At the beginning of the experiment, 10 unrelated healthy young adult albino guinea pigs of both sexes (5 males, 5 females) from a single strain weighing between 300 g and 500 g were used. After the animals were depilated 24 hours before the stimulation, the areas to be treated were shaved. The hair-free areas were thoroughly washed with warm water and dried with a towel before the animals were taken into the cages. After 2 hours of trimming the hairs, the test sites were graded according to the Magnusson and Kligman rating given in Chart 1.

CHART 1
Magnusson and Kligman rating scale

| Patch test reaction | Rating scale |
|---|---|
| No visible chance | 0 |
| Discrete or patchy erythema | 1 |
| Moderate and contiguous erythema | 2 |
| Prominent erythema | 3 |
| severe erythema and/or swelling | 4 |

Test material given under the title of ISO 10993-10:2010 annex A A.2,2 Liquid test materials; Liquids were applied without dilution or by direct precipitation or, if not applicable, by diluting with a suitable liquid at a dose of 1600 ppm, which is the usage dose according to the guideline. Saline solution-impregnated absorbent gauze was used as negative control. As a positive control, 0.8% paraformaldehyde solution is used in our laboratory.

The gauze impregnated with the inventive nanosolution was applied in direct contact with the upper left and lower right regions of the shaved area. The samples were thus kept in contact with the shaved back skin of the animal for 6 hours. The samples applied to the shaved area were covered with gauze and protected with plaster from the outside. This application was carried out three times a week for three weeks.

After three weeks of induction, after two weeks of rest, the samples were applied to the opposite sides of the previously induced areas for the stimulation experiment and kept for 6 hours. Evaluation was made at 24 and 48 hours following the stimulation application.

In the induction phase, observations were made according to Magnusson and Kligman grading at 24±2 and 48±2 hours following three applications per week for three weeks. Re-grading was performed 24±2 and 48±2 hours after the removal of the stimulation patch after a two-week interval.

Test and control animal observation records according to Magnusson and Kligman grading are given in Chart 2 below.

CHART 2
Magnusson and Kligman Rating in Groups

| | Induction first | | | Induction | | |
|---|---|---|---|---|---|---|
| | Application dates | | | | | |
| | 2020 Sep. 24 | 2020 Sep. 26 | 2020 Sep. 28 | 2020 Sep. 31 | 2020 Oct. 2 | 2020 Oct. 4 |
| 1. Negative | 0 | 0 | 0 | 0 | 0 | 0 |
| control | | | | | | |
| 2. Negative | 0 | 0 | 0 | 0 | 0 | 0 |
| control | | | | | | |
| 3. Negative | 0 | 0 | 0 | 0 | 0 | 0 |
| control | | | | | | |
| 4. Negative | 0 | 0 | 0 | 0 | 0 | 0 |
| control | | | | | | |
| 5. Negative | 0 | 0 | 0 | 0 | 0 | 0 |
| control | | | | | | |
| 1. Positive | 1 | 1 | 1 | 1 | 2 | 2 |
| Control | | | | | | |
| 2. Positive | 1 | 1 | 1 | 1 | 1 | 2 |
| Control | | | | | | |
| 3. Positive | 1 | 1 | 1 | 1 | 2 | 2 |
| Control | | | | | | |
| 4. Positive | 1 | 1 | 1 | 1 | 1 | 2 |
| Control | | | | | | |
| 5. Positive | 1 | 1 | 1 | 2 | 2 | 2 |
| Control | | | | | | |
| 1. Test | 0 | 0 | 0 | 0 | 0 | 0 |
| Animal | | | | | | |
| 2. Test | 0 | 0 | 0 | 0 | 0 | 0 |
| Animal | | | | | | |
| 3. Test | 0 | 0 | 0 | 0 | 0 | 0 |
| Animal | | | | | | |
| 4. Test | 0 | 0 | 0 | 0 | 0 | 0 |
| Animal | | | | | | |
| 5. Test | 0 | 0 | 0 | 0 | 0 | 0 |
| Animal | | | | | | |
| 6. Test | 0 | 0 | 0 | 0 | 0 | 0 |
| Animal | | | | | | |
| 7. Test | 0 | 0 | 0 | 0 | 0 | 0 |
| Animal | | | | | | |
| 8. Test | 0 | 0 | 0 | 0 | 0 | 0 |
| Animal | | | | | | |
| 9. Test | 0 | 0 | 0 | 0 | 0 | 0 |
| Animal | | | | | | |
| 10. Test | 0 | 0 | 0 | 0 | 0 | 0 |
| Animal | | | | | | |
| | Induction | | | Stim | 24th | 48th |
| | Application dates | | | | | |
| | 2020 Oct. 7 | 2020 Oct. 9 | 2020 Oct. 12 | 2020 Oct. 26 | 2020 Oct. 27 | 2020 Oct. 28 |
| 1. Negative | 0 | 0 | 0 | 0 | 0 | 0 |
| control | | | | | | |
| 2. Negative | 0 | 0 | 0 | 0 | 0 | 0 |
| control | | | | | | |
| 3. Negative | 0 | 0 | 0 | 0 | 0 | 0 |
| control | | | | | | |
| 4. Negative | 0 | 0 | 0 | 0 | 0 | 0 |
| control | | | | | | |
| 5. Negative | 0 | 0 | 0 | 0 | 0 | 0 |
| control | | | | | | |
| 1. Positive | 2 | 2 | 3 | 2 | 2 | 2 |
| Control | | | | | | |
| 2. Positive | 2 | 2 | 3 | 2 | 2 | 2 |
| Control | | | | | | |
| 3. Positive | 2 | 2 | 2 | 2 | 2 | 2 |
| Control | | | | | | |
| 4. Positive | 2 | 2 | 3 | 2 | 2 | 2 |
| Control | | | | | | |
| 5. Positive | 2 | 3 | 3 | 3 | 2 | 2 |
| Control | | | | | | |
| 1. Test | 0 | 0 | 0 | 0 | 0 | 0 |
| Animal | | | | | | |
| 2. Test | 0 | 0 | 0 | 0 | 0 | 0 |
| Animal | | | | | | |
| 3. Test | 0 | 0 | 0 | 0 | 0 | 0 |
| Animal | | | | | | |
| 4. Test | 0 | 0 | 0 | 0 | 0 | 0 |
| Animal | | | | | | |
| 5. Test | 0 | 0 | 0 | 0 | 0 | 0 |
| Animal | | | | | | |
| 6. Test | 0 | 0 | 0 | 0 | 0 | 0 |
| Animal | | | | | | |
| 7. Test | 0 | 0 | 0 | 0 | 0 | 0 |
| Animal | | | | | | |
| 8. Test | 0 | 0 | 0 | 0 | 0 | 0 |
| Animal | | | | | | |
| 9. Test | 0 | 0 | 0 | 0 | 0 | 0 |
| Animal | | | | | | |
| 10. Test | 0 | 0 | 0 | 0 | 0 | 0 |
| Animal | | | | | | |

In the sensitization test of the inventive nanosolution product using the Closed Patch-Buehler Test method, it was determined that the samples did not cause any appearance of erythema and edema, which is an indication of sensitivity/sensitivity in any of the subjects in the applications and observations made for 5 weeks.

In line with the directive of the TS EN ISO 10993-10:2010 standard, it was determined that the inventive liposomal ozone nanosolution did not have a delayed sensitizing effect as a result of the evaluation of our test observations with the Closed Patch-Buehler Experiment method.

Antivirus Efficacy Test of Liposomal Ozone Nanosolutions:

In order to investigate the biological effect and possible toxic effect of the inventive liposomal ozone nanosolution, epithelial cell culture was first performed. Therefore, the epithelial tissue piece obtained from a biobank was taken into saline (0.09% NaCl) after thawing and separated into small pieces in sterile petri dishes. Pre-prepared medium containing 10% fetal bovine serum and 1% antibiotic was cultured. After reaching sufficient maturity and saturation in cell culture dishes, they were left to incubate for varying times with varying doses of the product.

Tracheo-bronchial epithelial cells supplied and generally targeted by human respiratory system viruses were subjected to cell culture process by means of the bioreactor system in its infrastructure to provide an organotypic culture medium. In this process, after incubation with pre-prepared medium (DMEM) containing 10% fetal bovine serum and 1% antibiotic, in a 3-dimensional, closed system bioreactor containing 37° C. and 5% CO₂ for 48 hours, cell culture was continued for another 48 hours by washing with ready-made media. At the end of this period, cells were trypsinized with 0.25% trypsin-EDTA and passaged into other units of the bioreactor system that allow passage into the same medium. The culture was maintained by renewing the medium based on color and pH changes until the cells planted in these areas covered 80% of the surface area. When the cells reach the saturation level in their environment, the cells in a single chamber are exposed to trypsin-mediated treatment, then the 3-dimensional cell culture was separated from the surface and washed. In the remaining chambers, the active substance studies were continued with time and dose adjustments. As a result of the studies, its positive effect on cell proliferation was determined. This study was carried out using bioreactors and culture media that have the ability to imitate human nature.

After washing, cells were counted, and viability test was performed by flow cytometry. The resuspended cells were read in a 3-laser flow cytometer until there are 10000 cells and viability percentages of the cells obtained from the culture were determined by staining with the viability dye (7AAD). In addition, cultured cells at the level to be harvested were washed and taken into an eppendorf of 500 μL. 100 μL of 0.4% trypan blue solution was added to this cell suspension and incubated for 5 minutes at room temperature, then 10 μL was taken from thereof. Cell viability percentages were determined by placing this volume on the cell count slide and reading in a cell imaging and counting device.

As can be seen in the chart, the fact that neither proliferation nor viability is observed on healthy host cells is considered as a finding that the product will not cause a toxic effect.

| DOSE | INCUBATION | VITALITY % |
|---|---|---|
| Control-0 | 2 hours | 67 |
| Control-0 | 24 hours | 60 |
| 1600 ppm | 2 hours | 67 |
| 1600 ppm | 12 hours | 74 |
| 1600 ppm | 24 hours | 88 |
| 1000 ppm | 2 hours | 66 |
| 1000 ppm | 12 hours | 61 |
| 1000 ppm | 24 hours | 59 |
| 600 ppm | 2 hours | 68 |
| 600 ppm | 12 hours | 68 |
| 600 ppm | 24 hours | 67 |
| 300 ppm | 2 hours | 69 |
| 300 ppm | 12 hours | 68 |
| 300 ppm | 24 hours | 69 |

The effect of the active substance on the corona virus SARS COV-2 virus lysate was measured fluorometrically in a dose and time dependent manner in terms of both its effect on the initial volume and its effect on cDNA conversion, presented in the chart below. In the statistical analyzes performed with Student's t-test, the determination of statistically significant activity at 1000 ppm in a 5-minute incubation period is also in line with previous bacterial activity studies, it has been revealed that the product subject to the study can have both viral and bacterial activity.

| Initial | | | Final | |
|---|---|---|---|---|
| Concentration | | Incu- | Concentration | cDNA cons, |
| (fluorometer - | | bation | (fluorometer - | (fluorometer - |
| ng/ul) | Example | duration | ng/ul) | ng/ul) |
| 56.4 | CONTROL | — | 56.42 | 23.4 |
| 46.8 | 1600 | 30 sec. | 40.9* | 26.7* |
| 50.1 | | 1 min. | 22.5* | 17.8* |
| 38.8 | | 5 min. | 14.4* | 7.8* |
| 29.6 | 1000 | 30 sec. | 29.6 | 26.06 |
| 44.6 | | 1 min. | 42.4 | 25.6 |
| 71 | | 5 min. | 28.3* | 16.62* |
| 21.2 | 600 ppm | 30 sec. | 21.2 | 24.5 |
| 59 | | 1 min. | 59.6 | 23.9 |
| 50 | | 5 min. | 49.5 | 23.52 |
| 98.2 | 300 ppm | 30 sec. | 98.6 | 28.8 |
| 36.8 | | 1 min. | 36.8 | 26.56 |
| 28.8 | | 5 min. | 28.75 | 23.4 |

As a result of the studies detailed above, the inventive liposomal ozone nanosolution was concluded that it has both viral and bacterial activity found; does not cause toxic effects; does not have a negative effect on the viability of healthy cells; has both regenerative and proliferative effects on human respiratory system (tracheo-bronchial) cells.

—Test Analysis Results of Injection Solutions—

In Vitro Genotoxicity Bacterial Ames Test of Liposomal Ozone Nanosolutions:

Genotoxicity tests were performed on the samples of liposomal ozone nanosolutions of the invention for Intra-articular/Subcutaneous/Intramuscular Injection and has been shown to have no mutagenic potential.

Genotoxicity (Bacterial Ames Test) (OECD 471) Information: Studies for the development of various test systems to elucidate the mutagenic effect and the detection of mutagens and to reduce the risk of hereditary diseases and cancers caused by mutagenesis for humans constitute one of the most important research areas of genetic toxicology. The Ames test, which was developed by Bruce Ames in 1972 and applied as a screening test to determine the mutagenic effects of chemical substances, is widely and reliably used as a short-time bacterial test system.

One of the short-time test systems used to determine the mutations caused by chemicals at the cell level is the Ames test. The Ames test is considered as one of the reliable methods to examine the toxic, mutagenic-carcinogenic effects of test substances, which are intended to be used as pharmaceutical raw materials.

Preparation of Test Substance and Control Substance:

A. For solid test material: According to OECD 471 guidelines, 0.5 g of test product was dissolved in 10 mL of DMSO, vortexed, and a 2-fold serial dilution was made. Final concentrations of the test were adjusted to be 5 mg/plate, 2.5 mg/plate, 1.25 mg/plate, 0.625 mg/plate, and 0.3125 mg/plate.

B. For liquid test material: According to OECD 471 guidelines, 50 μL of test product was dissolved in 1 mL of DMSO, vortexed, and a 2-fold serial dilution was made. Final concentrations of the assay were adjusted to be 5 μL/plate, 2.5 μL/plate, 1.25 μL/plate, 0.625 μL/plate, and 0.3125 μL/plate.

Strains used in the study: All strains were purchased from MOLECULAR TOXICOLOGY, INC. Strain numbers are shown below:

a. Salmonella typhimurium TA98: #71-098L

b. Salmonella typhimurium TA100: #71-100L

Positive Control: Different positive control products are used based on different bacterial strains. Description of bacterial strains and individual concentrations of mutagens are shown below:

| Positive control chemicals | Solvent | Bacterial strains |
|---|---|---|
| 2-aminoanthracene 100 μg | DMSO | Salmonella strains + |
| | | S9 |
| 4-nitroquinoline-N-oxide 50 μg | DMSO | TA100 |
| N-Aminocytidine 2.5 mg | Distilled water | TA100 |
| 2-Nitrofluorene 50 μg | DMSO | TA98 |

Negative Control: DMSO

For the metabolic activation system (S9 mix), Arachlor 1254-induced lyophilized rat liver S9 microsomal fraction was used. The mutagenic potential of the extracts was evaluated in the presence and absence of the S9 mixture. A 30% S9 mixture was used in the tests.

Bacteria were incubated 1 night before inoculation on ampicillin medium. Control solutions were prepared separately for each bacterium in 1 ml of the appropriate solvent (DMSO or sterile distilled water) specified in the Control Chart. Six concentrations were determined for the test material.

Prepared culture solutions were added to 384 wellplates. According to the test protocol, it was incubated for 48 hours in a 37-degree oven. At the end of the period, 64 wells per concentration were counted and evaluated according to positive [(yellow-revertant)] and negative [(purple-non-revertant)] wells the test was considered as positive (mutation effect) if two times more mutations were observed in the test strains with and without S9 than in the negative control group.

Genotoxicity (Bacterial Ames Test) Result

CHART 1
Demonstration of mutant colony numbers of
bacterial strains in S9 and S9-free medium of test material

| Test Strains | | | TA98 | | TA100 | |
|---|---|---|---|---|---|---|
| S9 | | | + | − | + | − |
| Mutant | Negative control | | 10 | 9 | 8 | 12 |
| Colony | Positive Control | | >48 | >52 | >45 | >44 |
| numbers | Test | 5 | 18 | 17 | 15 | 12 |
| | material | 2.5 | 15 | 15 | 14 | 11 |
| | concentrations | 1.25 | 12 | 14 | 14 | 12 |
| | μl/plate | 0.625. | 10 | 13 | 13 | 9 |
| | | 0.3125 | 10 | 11 | 13 | 10 |

Comment: It was observed that the colonies of the “Intra Articular/Subcutaneous/Intramuscular Injection Solution” test product, Salmonella typhimurium TA98, TA100, did not appear to be 2 times higher than the colonies of the negative control group.

As a result, it was determined according to the results of the test procedures (OECD 471) that the test material was not mutagenic at all lower concentrations with 2 different bacterial strains.

Liposomal Ozone Nanosolutions Acute Systemic Toxicity Test (TS EN ISO 10993-11:2018):

Acute systemic toxicity test was performed on the samples of liposomal ozone nanosolutions for Intra-Articular/Subcutaneous/Intramuscular Injection of the invention and it was determined that there was no acute toxicity effect.

Acute systemic toxicity test was carried out considering the TS EN ISO 10993-11:2018 standard, the experimental animal used in the test is considered according to S EN ISO 10993-2:2006 standard, the material preparation is made according to TS EN ISO 10993-12:2012 standard guidelines.

The test material was carried out in accordance with the TS EN ISO 10993-12:2012 sample preparation guide. Intra-Articular/Subcutaneous/Intramuscular Injection nanosolution was injected intramuscularly at an amount of 2 ml/kg body weight (TS EN ISO 10993-11:2018-4, Appendix B).

Liposomal ozone nanosolution was injected intramuscularly into the gluteal region of the subjects at an amount of 2 ml/kg body weight. As the control group, 6 (3 ♀/3 ♂) BalbC mice were injected with saline in the amount of 2 ml/kg body weight and the control group was formed. The control group subjects were kept in the same environment with the experimental group and fed in the same way. Subjects were kept under clinical observation for three days (Chart 1).

Clinical Observation:

Body weight: The body weight of the subjects was in the range of 23-33 g, and there was no remarkable change in weights.

| | Starting | Day 1 | Day 2 | Day 3 |
|---|---|---|---|---|
| | Weight | Weight | Weight | Weight |
| | (gr) | (gr) | (gr) | (gr) |
| Control group | | | | |
| 1st Subject ( [symbol] ) | 23 | 23 | 24 | 24 |
| 2nd Subject ( [symbol] ) | 24 | 25 | 24 | 25 |
| 3rd Subject ( [symbol] ) | 25 | 25 | 25 | 25 |
| 4th Subject ( [symbol] ) | 31 | 31 | 32 | 31 |
| 5th Subject ( [symbol] ) | 31 | 32 | 32 | 32 |
| 6th Subject ( [symbol] ) | 32 | 32 | 32 | 32 |
| Experimental Group ( [symbol] ) | | | | |
| 1st Subject ( [symbol] ) | 32 | 32 | 32 | 31.5 |
| 2nd Subject ( [symbol] ) | 32 | 31 | 31 | 31 |
| 3rd Subject ( [symbol] ) | 32 | 32 | 33 | 32 |
| 4th Subject ( [symbol] ) | 33 | 32 | 32 | 32 |
| 5th Subject ( [symbol] ) | 33 | 33 | 34 | 34.5 |
| 6th Subject ( [symbol] ) | 33 | 33 | 32 | 32.5 |
| Experimental Group ( [symbol] ) | | | | |
| 1st Subject ( [symbol] ) | 26 | 26 | 27 | 26 |
| 2nd Subjects ( [symbol] ) | 29 | 29 | 29 | 29 |
| 3rd Subject ( [symbol] ) | 30 | 30 | 30 | 30 |
| 4th Subject ( [symbol] ) | 30 | 30 | 31 | 30 |
| 5th Subject ( [symbol] ) | 31 | 30 | 31 | 31 |
| 6th Subject ( [symbol] ) | 31 | 31 | 31 | 31 |

[symbol]

indicates data missing or illegible when filed

CHART 1
Common clinical signs, observations.

| Clinical | Observation Signs | Affected system | Evaluation |
|---|---|---|---|
| Respiratory | Shortness of breath | CNS (Central | Not seen |
| | (abdominal breathing, | Nervous System), | |
| | difficulty breathing). apnea, | Lung, heart | |
| Motor | Increased/decreased | CNS, | Not seen |
| movements | somnolence, loss of righting, | somatomotor, | |
| | anesthesia, catalepsy, | sensory, | |
| | exhaustion shivering, | | |
| | twitching (fasciculation) | | |
| Convulsion | Clonic, tonic, tonic-clonic, | CNS, | Not seen |
| | asphyxial, opistotonus | neuromuscular, | |
| Reflexes | Corneal, righting, myotact, | CNS: sensory, | Normal |
| | light, startle reflex | herbaceous, | |
| Ocular symptoms | Lacrimation (tears), | Autonomous, | Not seen |
| | miosis, mydriasis, | irritation | |
| | exoltalmus, ptosis, opacity, | | |
| | iris inflammation, | | |
| | conjunctivitis, | | |
| Cardiovascular | Bradycardia, tachycardia, | CNS, | Not seen |
| symptoms | arrhythmia | autonomous, | |
| Salivation | Excessive | Autonomous | Not seen |
| Pyloerection | Bristle | Autonomous | Not seen |
| Muscle tone | Hypotonia, hypertonia | Autonomous | Normal tone |
| Gastrointestinal | Soft stools, diarrhea, nausea, | CNS, | Not seen |
| | diuresis. Rhinorrhea | herbaceous, | |
| | | sensory, | |
| | | Gastrointestinal | |
| Skin | Edeme Erythema | Tissue damage, | Not seen |

Activation: No changes were observed in the motility and daily activations of the subjects. Increasing and decreasing drowsiness, fatigue, trembling, twitching behaviors were not observed.

Stress: In the behavior of the subjects, neurotic behaviors such as stress indication, constant touring, aggression or cornering were not observed. They behaved in normal activity in the cage.

Death: No deaths were observed in any group.

Pain: Passivation, crawling, intermittent mobility, and shouting behavior were not observed as pain indicators.

Respiration: No difference in respiratory behavior and no cyanosis, nasal discharge, tachypnea was observed.

Food and water consumption: Food and water consumption was monitored similarly to controls.

Sight: Conjunctival hyperemia, lacrimation, conjunctivitis, opacification, iris inflammation was not observed in the eyes.

Cardiac observation: Tachycardia, bradycardia was not observed.

Body temperature: 36.5° C. was observed at a normal level.

Gastrointestinal observation: Excessive salivation, diarrhea, soft stools were not observed. No evidence of toxicity was found during the experiment, in acute applications of Intra-Articular/Subcutaneous/Intramuscular Injection liposomal ozone nanosolution, which was tested with clinical observations and measurements according to the protocol and evaluation criteria specified in the TS EN ISO 10993-11:2018 document. As a result, it was determined that liposomal ozone nanosolution for Intra-Articular/Subcutaneous/Intramuscular Injection did not have any acute toxicity effect.

In Vitro Cytotoxicity Test of Liposomal Ozone Nanosolutions (TS EN ISO 10993-5:2009):

In Vitro Cytotoxicity test was performed on the liposomal ozone nanosolution samples of the invention for Intra-Articular/Subcutaneous/Intramuscular Injection and it was revealed that it did not have cytotoxic potential.

Extraction of test material, negative and positive groups was carried out in accordance with ISO 10993-12 standards. The extract was prepared by keeping 0.2 grams/ml of test material in DMEM 10% FBS for 72±2 hours at 37±1° C. in a shaking incubator at 200 rpm (The specified standard is based on irregular shaped solid devices. This case is chosen as the worst-case scenario). It was filtered through a 0.22 μm sterile filter and defined as 100% extract. Percentages of other extracts were formed by dilution of DMEM 10% FBS. No color change was observed (In-lab test method).

L-929 mouse fibroblast cell line from ATCC for cytotoxicity test study (NCTC clone 929: CCL 1, American Type Culture Collection [ATCC] was used. Cells were propagated in DMEM (dulbecco's modified eagle medium) (ATCC Cat No: 30-2006) medium supplemented with 10% FBS (fetal bovine serum) and 2% glutamine and incubated at 37t in an oven with 5% CO2. A mixture of 0.25% trypsin and 0.03% EDTA was used for trypsinase of cells as recommended by ATCC. Cells were suspended in culture medium and 100 μl was transferred to 96-well plates with 104 cells in each well.

After 24 hours of cell culture conditions, the medium on the culture was removed and 100 μl of test material was added from the positive and negative control extracts. All doses were administered with at least 5 repetitions. At the end of 48 hours of waiting in an oven with 5% CO2 at 37° C. and 95% humidity, the culture medium was removed after the plates were examined.

Negative control (NC): Cell culture medium (DMEM+10% FBS) was incubated under the same conditions as the extracts for 72 hours.

Positive Control (PC): Serial dilutions of DMSO (Dimethylsulfoxide) (10-30 v/v) were made with DMEM+10% FBS.

Test Material (TM) concentrations: It was diluted with DMEM+10% FBS as 100-30-10-3-v/v.

After microscopic examination of the plates, the culture medium was removed from the wells. 50 μl of MTT solution was added to one of its test wells. The plates were incubated for 2 hours at 37° C. Then, the MTT solution was re moved from the wells and 100 μl of isopropanol was added to each well. Absorbance measurements were made and evaluated with a microplate reader containing a 570 nm filter. Absorbances greater than 0.2 were accepted as the general validity criterion of the test. If the viability of the test material is less than 70%, it is considered cytotoxic.

CHART 1
Results of quantitative measurement of cytotoxic effects by MTT Test.

| | optical density (OD) mean value | | | | | | | |
|---|---|---|---|---|---|---|---|---|
| | 570 nm ± standard deviation (SD) | | | | Vitality % | | | |
| | % Dilution concentrations (v/v) | | | | % Dilution concentrations (v/v) | | | |
| | 100 | 30 | 10 | 3 | 100 | 30 | 10 | 3 |
| TM | 0.21 ± 0.014 | 0.21 − 0.02 | 0.23 ± 0.09 | 0.235 ± 0.05 | 84 | 84 | 92 | 94 |
| NC | 0.25 ± 0.05 | | | | 100 | | | |
| PC | | 0.15 ± 0.02 | 0.22 ± 0.01 | | | 36.46 | 53.44 | |

| | |
|---|---|
| BLANK | 0.046 ± 0.002 |

It has been determined that the test material does not have a cytotoxic effect according to the test results carried out in line with the directive of TS EN ISO 10993-5 standard.

Irritation Test Result of Liposomal Ozone Nanosolutions in TS EN ISO 10993-10:2010 Standards

Irritation test on liposomal ozone nanosolution samples of the invention for Intra-Articular/Subcutaneous/Intramuscular Injection was carried out considering TS EN ISO 10993-10:2010 standard, the experimental animal used in the test is considered according to TS EN ISO 10993-2:2006 standard, the material preparation is made according to TS EN ISO 10993-12:2013 standard guidelines.

Three healthy young New Zealand albino rabbits with a body weight of 2-3 kg were used as experimental animals.

Test material given under the title of TS EN ISO 10993-10:2010 annex A A.2,2 Liquid test materials; Liquids should be tested without dilution or by direct precipitation or, if not applicable, by diluting with physiological saline to ¼ dose, which is the usage dose in accordance with the directive that it should be tested by diluting with a suitable liquid.

Sodium lauryl sulphate (SLS) was used as a positive control and distilled water was used as a negative control.

The sponge impregnated with liposomal ozone nanosolution was kept in direct contact with the sample application areas no 2 as shown in FIG. 2. SLS-impregnated sponge was applied to the positive control area (area no 3). The samples were covered with gauze, fixed with a bandage, and topically contacted with the back skin for 4 hours.

The primary irritation index was determined by evaluating the application areas (1±0.1) s, (24±2) s, (48±2) s, and (72±2) hours after the 4th hour application, according to the skin reaction score given in Chart 1. As stated in the standard, the 1st hour was not included in the calculation.

The observations made according to the TS EN ISO 10993-10:2010 standard and their evaluations are given in Chart 1 and Chart 2.

CHART 1
Irritation score

| | | | Observation | | | | | | | |
|---|---|---|---|---|---|---|---|---|---|---|
| | | | Erythema | | | | Edema | | | |
| Animal | | Application | 1st | 24th | 48th | 72nd | 1st | 24th | 48th | 72nd |
| No | Groups | areas | hour | hour | hour | hour | hour | hour | hour | hour |
| 1 | Sample | Left front | 1 | 0 | 0 | 0 | 0 | 0 | 0 | 0 |
| | | Right back | 1 | 0 | 0 | 0 | 0 | 0 | 0 | 0 |
| | Positive | Right front | 4 | 4 | 3 | 1 | 3 | 2 | 2 | 1 |
| | Control | | | | | | | | | |
| | Negative | Left back | 0 | 0 | 0 | 0 | 0 | 0 | 0 | 0 |
| | control | | | | | | | | | |
| 2 | Sample | Left front | 1 | 1 | 0 | 0 | 0 | 0 | 0 | 0 |
| | | Right back | 0 | 0 | 0 | 0 | 0 | 0 | 0 | 0 |
| | Positive | Right front | 3 | 3 | 2 | 1 | 3 | 2 | 0 | 0 |
| | Control | | | | | | | | | |
| | Negative | Left back | 0 | 0 | 0 | 0 | 0 | 0 | 0 | 0 |
| | control | | | | | | | | | |
| 3 | Sample | Left front | 0 | 0 | 0 | 0 | 0 | 0 | 0 | 0 |
| | | Right back | 0 | 0 | 0 | 0 | 0 | 0 | 0 | 0 |
| | Positive | Right front | 3 | 3 | 2 | 1 | 2 | 2 | 1 | 0 |
| | control | | | | | | | | | |
| | Negative | Left back | 0 | 0 | 0 | 0 | 0 | 0 | 0 | 0 |
| | control | | | | | | | | | |

CHART 2
Score average

| | | | | Primary |
|---|---|---|---|---|
| | Primary irritation score | | | Index |
| Example | Rabbit 1 | Rabbit 2 | Rabbit 3 | Irritation |
| Sample | 0.0 ± 0.0 | 0.083 ± 0.289 | 0.0 ± 0.0 | 0.027 |
| Positive | 2.167 + 1.169 | 1.333 ± 1.211 | 1.500 ± 1.049 | 1.666 |
| Control | | | | |
| Negative | 0.0 ± 0.0 | 0.0 + 0.0 | 0.0 ± 0.0 | 0.0 |
| control | | | | |

It was determined that liposomal ozone nanosolution for Intra-Articular/Subcutaneous/Intramuscular Injection did not have any irritating effect according to the test results carried out in line with the directive of the TS EN ISO 10993-10:2010 standard.

Subcutaneous Implantation 7-14 Days Test of Liposomal Ozone Nanosolutions (TS EN ISO 10993-6:2016)

Subcutaneous Implantation test was performed on the inventive liposomal ozone nanosolution samples for Intra-Articular/Subcutaneous/Intramuscular Injection and it has been shown that it does not cause any inflammatory reaction.

The purpose of the test method is to determine the history and course of the tissue response, which includes the eventual integration or resorption/degradation of the material after medical device/biomaterial implantation. Particularly, the degradation properties and texture response of the material are determined for degradable/absorbable materials.

Implants are tested by placing them in the back subcutaneous tissue of adult mice, rats, guinea pigs or rabbits.

Healthy Wistar rats weighing 200-300 g and 10 weeks old of both sexes were used in the test studies. The subjects were kept in a sheltering area with 60% humidity and a constant temperature of 22° C. during the test. The subjects were given normal rat feed and tap water. Dust-free autoclaved wood shavings were used as substrate. At the end of the experiment, all animals were euthanized.

Liposomal ozone nanosolution samples could not be applied directly under the skin (implantation). Product implantation was performed under operating room conditions by placing rats anesthetized at a dose of Ketamine 85 mg/kg, Xylazine 15 mg/kg body weight, under the dorsal skin on either side of the spine. After the hairs were removed by shaving the operation area, it was cleaned by wiping with an antiseptic solution. The implantation period was determined as 2 weeks, and at the end of the process, the subjects were euthanized using deep anesthesia, and the implantation areas were opened, and macroscopic observation was made. Tissue samples dissected from the implantation area were taken for microscopic examination.

The treated animals did not show any abnormalities in their daily behavior and activation. No abnormality was observed in its movements and walk. Their eating habits and social behavior in the cage were normal.

At the end of the fourteen-day implantation period, it was observed that the Intra-Articular/Subcutaneous/Intramuscular Injection nanosolution product was absorbed under the skin and disappeared in the hypodermis in the macroscopic examination in the implantation area. No lymph node formation and inflammatory structure were observed around the implantation focus. No lymphatic focus or extra lymphocyte infiltration and metaplasic changes were observed at the implantation site in hypodermal tissues in the histopathological examination.

According to the protocol and evaluation criteria specified in the TS EN ISO 10993-6: 2016 document, it was observed that the Intra-Articular/Subcutaneous/Intramuscular Injection Nanosolution was completely absorbed and lost, and did not cause any inflammatory or metaplasic changes in harmony with the subcutaneous tight connective tissue in the implantation area in the 14-day subcutaneous implantation application as a result of the implantation experiments of the Intra-Articular/Subcutaneous/Intramuscular Injection nanosolution product tested with the observations and measurements.

Consequently, following 14 days of subcutaneous implantation of Intra-Articular/Subcutaneous/Intramuscular Injection Nanosolution, it was determined that it was completely absorbed without leaving any residue in the implantation area and integrated with the surrounding tissue and did not cause any inflammatory reaction.

Intramuscular Implantation Test (TS EN ISO 10993-6:2016) of Liposomal Ozone Nanosolutions:

Intramuscular Implantation test of liposomal ozone nanosolution samples of the invention for Intra-Articular/Subcutaneous/Intramuscular Injection was performed and it has been shown that it does not cause any deformative or inflammatory effects in muscle tissue.

The aim of the test method is to determine the date and course of the tissue response, which includes the eventual integration or resorption/degradation of the material after implantation of the medical device/biomaterial. Particularly, the degradation properties and texture response of the material are determined for degradable/absorbable materials. Implants should be inserted into the Panniculus carnosa or gluteal region muscles of adult mice, rats, guinea pigs or rabbits. Implant specimens should be implanted aseptically and without any damage prior to or during implantation. A minimum of three animals and sufficient space are used to obtain a total of 10 test and 10 control specimens for each material and implantation period. When multiple tissue samples are taken from a single implant site, the histology sections should be at least 1 cm apart.

In the test studies, 6 (3 female/3 male) BALB/c mice weighing 17-20 g were used as subjects. The subjects were kept in a sheltering area with 60% humidity and a constant temperature of 22° C. during the test. The subjects were given normal rat feed and tap water. Dust-free autoclaved wood shavings were used as substrate. At the end of the experiment, all animals were euthanized.

2 ml of Intra-Articular/Subcutaneous/Intramuscular Injection Solution was injected into the right and left gluteal muscles of the subjects. 14 days after the injection, the gluteal muscles of the right and left legs were dissected and examined microscopically from the subjects who were anesthetized with Ketamine 85 mg/kg and Xylazine 15 mg/kg.

The treated animals did not show any abnormalities in their daily behavior and activation. No abnormality was observed in its movements and walk. Their eating habits and social behavior in the cage were normal.

At the end of the 14-day implantation period, after ether anesthesia, cervical dislocation was applied to the subjects, and injection sites were opened in the gluteal muscles of the hind extremities. Injection areas were examined macroscopically. No residues related to Intra-Articular/Subcutaneous/Intramuscular Injection Nanosolution were found. In addition, lymphatic focus formation due to inflammation in the muscle was not observed.

When the gluteal regions where liposomal ozone nanosolution was injected were examined; it was observed macroscopically that the muscle structures have a normal appearance. It was observed during the dissection of the muscle that there were no different tissue masses or lymphatic foci within the muscle. It was observed that the liposomal ozone nanosolution injected into the muscle tissue was completely absorbed by the tissue and did not leave any residue. It was observed that the injection of liposomal ozone nanosolution in the muscle tissue did not cause any negative structural changes in the tissue.

It was determined that lysosomal ozone nanosolution samples for Intra-Articular/Subcutaneous/Intramuscular Injection did not cause any deterioration or deformation in the muscle tissue, and lymphocyte infiltration in the muscle during microscopic examinations.

The intramuscular implantation test of the liposomal ozone nanosolution product was performed according to the protocol and evaluation criteria specified in the TS EN ISO 10993-6:2016 document. Intra-Articular/Subcutaneous/Intramuscular Injection product at the end of the 2-week gluteal experiment period, it was determined that the samples were completely absorbed and there was no residue left in observation and evaluations. It was determined macroscopically and microscopically that the injection was in harmony with the muscle tissue and did not cause deformation and inflammatory reaction in the muscle structure.

As a result, it was determined that intramuscular implantation applications of liposomal ozone nanosolution product for Intra-Articular/Subcutaneous/Intramuscular Injection did not cause any deformative or inflammatory effect on muscle tissue.

Liposomal Ozone Nanosolution Biopsy+Histopathology/Hematoxylin Eosin Staining Test:

In the liposomal ozone nanosolution samples of the invention for Intra-Articular/Subcutaneous/Intramuscular Injection, in an experimental study on rats, 280 skin biopsy materials from 56 female rats of Sprague downey breed, 2 months old, were euthanized at the end of the experiment, microscopic examinations of the tissues of the lungs, liver and kidneys were made so as to observe the “Healing Effects in Regional Subcutaneous Applications”.

Applying physiological salt water, 10 ppm, 50 ppm, 100 ppm, 500 ppm, 1000 ppm, 5000 ppm subcutaneously to the experimental groups consisting of 56 animals, from the back region of the animal in each group, a total of 280 skin biopsies were examined followed by the 1st, 2nd, 3rd, 4th and 30th days. Lesions were scored and group average scores were obtained.

In the skin biopsies examined; it was observed that in the ratio of skin to epidermis (%) average of the first 4 days; 28.6 at a dose of 10 ppm, 48.3 on the 30th day; while it was 29.4 at a dose of 50 ppm, it was 42.8 on the 30th day; while it was 28.6 at a dose of 100 ppm, it was 33 on the 30th day; while it was 30.1 at a dose of 500 ppm, it was 455.6 on the 30th day; while it was 26 at 1000 ppm dose, it was 31.7 on the 30th day; while it was 34.1 at 5000 ppm dose, it was 27.1 on the 30th day.

The number of vascularization (in pieces) average of the first 4 days was observed as follows; while it was 7 at a dose of 10 ppm, it was 8.57 on the 30th day; while it was 6.83 at a dose of 50 ppm, it was 6.83 on the 30th day; while it was 5.33 at a dose of 100 ppm, it was 3.83 on the 30th day; while it was 6.12 at a dose of 500 ppm, it was 6.25 on the 30th day; while it was 4.84 at a dose of 1000 ppm, it was 4.57 on the 30th day; while it was 5.12 at 5000 ppm dose, it was 6.87 on the 30th day.

Hair follicle (in number) average of the first 4 days was observed as follows; while it was 16.5 at a dose of 10 ppm, it was 31.25 on the 30th day; while it was 12 at a dose of 50 ppm, it was 21.33 on the 30th day; while it was 25.17 at a dose of 100 ppm, it was 35.5 on the 30th day; while it was 24 at a dose of 500 ppm, it was 55.5 on the 30th day; while it was 13.43 at a dose of 1000 ppm, it was 32.28 on the 30th day; while it was 24.75 at 5000 ppm dose, it was 38.38 on the 30th day.

In general, no increase in neutrophil counts in the skin was observed in the whole day and groups. No pathological findings were found in the lung, liver, or kidney.

Microscopic examinations of the tissues of 72 skin biopsy materials belonging to 72 female rats of Sprague downey breed which were 2 months old, were performed in the experimental study on rats for “Investigation of the wound effects after injection into the Laparotomy Incision Line from clinical and histopathological aspects” with the liposomal ozone nanosolution samples of the invention for Intra-articular/Subcutaneous/Intramuscular Injection.

A total of 72 skin biopsies of 72 animals from the abdomen were examined in the experimental groups in the applications on the wound lips on the skin line where the incision was made; Physiological Salt Water (FTS), 1% Lidocaine, ozone-free nanosolution, 250 ppm ozone nanosolution, 500 ppm ozone nanosolution, 500 ppm ozone nanosolution+2% Lidocaine (50% blend), 1000 ppm Ozone nanosolution+2% Lidocaine (50% blend) and 2000 ppm ozone nanosolution+2% Lidocaine (50% blend). Lesions were scored and group average scores were obtained.

Microscopic Findings: * Scoring values out of 3 in examined skin biopsies are presented in the chart below.
*0: Negative, 1: mild. 2. Moderate, 3: Severe

| | | | | | | Collagen |
|---|---|---|---|---|---|---|
| | Connective | Vascular | | | | Epidermis/ |
| Group | tissue | formation | Inflammation | Edema | Bleeding | Dermis (%) |
| Physiological | 1.17 ± 0.60 | 1.83 ± 0.40 | 1.00*0.37 | 0.17 ± 0.17 | 0.33 ± 0.21 | 29.60 ± 5.38 |
| saline solution | | | | | | |
| 1% lidocaine | 2.67 ± 0.33 | 1.83 ± 0.17 | 2.50 ± 0.34 | 0.67 ± 0.33 | 0.83 ± 0.54 | 25.33 ± 2.46 |
| Ozone-free solution | 2.13 ± 0.23 | 2.13 ± 0.23 | 1.38 ± 0.32 | 0.63 ± 0.32 | 0.13 ± 0.13 | 27.00 ± 3.96 |
| 250 ppm | 2.86 ± 0.14 | 2.57 ± 0.20 | 2.43 ± 0.29 | 1.00 ± 0.38 | 0.36 ± 0.26 | 24.00 ± 4.43 |
| ozone solution | | | | | | |
| 500 ppm | 2.38 ± 0.13 | 2.00 ± 0.27 | 1.63 ± 0.18 | 1.88 ± 0.35 | 0.50 ± 0.38 | 32.83 ± 6.01 |
| ozone solution | | | | | | |
| 1000 ppm | 2.00 ± 0.13 | 1.50 ± 0.29 | 2.25 ± 0.48 | 1.00 ± 0.41 | 0.00 ± 0.00 | 52.50 ± 16.52 |
| ozone solution | | | | | | |
| 500 ppm ozone + | 3.00 ± 0.00 | 2.67 ± 0.21 | 1.83 ± 0.17 | 1.17 ± 0.31 | 0.67 ± 0.33 | 27.80 ± 1.36 |
| 2% lidocaine | | | | | | |
| 50% blend | | | | | | |
| 1000 ppm ozone + | 2.38 ± 0.26 | 2.25 ± 0.31 | 2.00 ± 0.27 | 1.63 ± 038 | 1.00 ± 0.27 | 40.50 ± 5.B1 |
| 2% lidocaine | | | | | | |
| 50% blend | | | | | | |
| 2000 ppm ozone + | 3.00 ± 10.00 | 2.00 ± 0.27 | 1.75 ± 0.16 | 0.75 ± 0.25 | 0.50 ± 0.27 | 30.00 ± 8.41 |
| 2% lidocaine | | | | | | |
| 50% blend | | | | | | |

In the Intra-Articular/Subcutaneous/Intramuscular Injection nanosolution samples of the invention, for observing “Systemic Effects from Regional Subcutaneous Applications”, as a result of euthanasia of 6 Albino laboratory rabbits, microscopic examinations of the tissues of the lungs, liver, heart muscle and kidneys and peripheral smear were performed in the study performed on rabbits and the results are presented below.

Macroscopic Findings: Animals were euthanized after 10 days of repeating 100 ml serum containing 200 ppm (6 mg/kg) ozone nanosolution intravenously.

Microscopic Findings: No pathological findings were found in the peripheral blood smears of the same animals, in the membrane structures and morphologies of erythrocytes; as well as in the lung, liver, heart muscle and kidney tissues.

In the liposomal ozone nanosolution samples of the invention for Intra-articular/Subcutaneous/Intramuscular Injection, for “Clinical and histopathological investigation of the curative effects of regional intra-knee injection; Comparison of knees with hyaluronic acid and nanoparticle vitamin injections”, in the experimental study on rats, microscopic examinations of bone and cartilage tissues of 64 female rats of Sprague downey breed, 2 months old, were made and the report is presented below.

Macroscopic Findings: After the defect previously performed with a 250 micrometer stopper eye scalpel to the medial condyle, disarticulation at the 6th week resulted in knee joint insertion, Serum Physiological, of Hyaluronic (HA), 500 ppm intra-articular, 1000 ppm intra-articular, 2000 ppm intra-articular, 1000 ppm+HA (1/2 ratio mixture) intra-articular, 2000 ppm+HA (1/2 ratio mixture) intra-articular, 4000 ppm+HA (1/2 mixture) namely a total of 64 tissues were examined from the joint region of 8 animals.

| Animal | Physiological | | | | | 1000 | 2000 | 4000 |
|---|---|---|---|---|---|---|---|---|
| number/ | saline | Hyaluronic | | 1000 ppm | 2000 ppm | ppm + HA | ppm + HA | ppm + HA |
| Score | solution | acid (HA) | 500 ppm | intraarticular | intraarticular | (1/2 ratio) | (1/2 ratio) | (1/2 ratio) |
| 1 | 6 | 3 | 1 | 1 | 1 | 1 | 1 | 1 |
| 2 | 3 | 1 | 1 | 2 | 1 | 0 | 0 | 0 |
| 3 | 7 | 4 | 0 | 0 | 2 | 2 | 0 | 1 |
| 4 | 4 | 2 | 1 | 2 | 2 | 1 | 1 | 1 |
| 5 | 6 | 4 | 2 | 1 | 1 | 0 | 0 | 0 |
| 6 | 5 | 2 | 1 | 0 | 2 | 1 | 1 | 1 |
| 7 | 5 | 4 | 2 | 1 | 0 | 1 | 1 | 1 |
| 8 | 4 | 4 | 2 | 2 | 2 | 1 | 1 | 1 |
| Average | 5.375 | 3 | 1.25 | 1.125 | 1.375 | 0.875 | 0.625 | 0.75 |
| score | | | | | | | | |

Microscopic Findings: Semi-quantitatively, the modified form of the scores of the studies of Pineda et al. (1992)* and Wakitani et al. (1994)** was used.

—Test Analysis Results of Herbal Solutions—

Laboratory Test on the Effect of Liposomal Ozone Nanosolutions on the Biological Activities of Harmful Carpoglyphus lactis (L.) (Acari: Carpoglyphidae) in Dried Apricots:

Carpoglyphus lactis (L.) (Astigmata: Carpoglyphidae), an important mite pest of dried fruits, causes the product to deteriorate, release unpleasant odors and eventually lose its market value when they are fed with fruit sugars of the dried apricots. Previous studies have shown that ozone gas can be used for control purposes in dried fruits due to its poisonous property against mites. However, due to the rapid decomposition of ozone gas into O2 and O—, its short persistence limits the use of this gas in mite control. The biological activities of the inventive liposomal ozone nanosolutions on this harmful mite were investigated in the study carried out within the scope of the invention. The lethality [lethal concentrations (LC), lethal time (LT)], repellent and egg laying inhibitor effects and persistence of the formulation on mites were evaluated in the study. This study showed that 0.27% concentration of the formulation killed 90% of female mites within 48 hours. According to the results of the probit analysis, lethal time (LT90) was determined as 121, 81.5 and 66 hours, respectively in case the liposomal ozone nanosolution of the invention is used against mites at rates of 0.1, 0.2 and 0.4%. Persistence tests showed that there was no statistically significant change in the lethal effects of the formulation during the first 7 days. However, after 10 days, a decrease of 12-17% in its lethal effect was detected. According to Pearson's chi-square test for evasive effect, strong escapes of C. lactis females were observed from apricots sprayed with concentrations above 0.1% of the formulation. In conclusion, it was determined with this study that the determined doses of the nanosolutions of the invention had toxic and repellent effects on C. lactis females. It has been demonstrated by this study that the formulation can be used in the control of C. lactis if the dried apricots are exposed to the doses determined here, with the persistence of the formulation for 10 days.

Laboratory Test on Toxic Effect of Liposomal Ozone Nanosolutions on Bacterial Crown Gall Nematode (Meloidogyne sp.):

The use of the inventive liposomal nanosolutions in the field of Plant Protection was investigated with three different samples containing Ozone (1), Ozone+Acetic Acid (2), Ozone+Acetic Acid+Tymol (3) so as to investigate the possibilities of use in agriculture. Bacterial crown gall nematodes, which cause crop losses in agriculture and have limited means of struggle, have been discussed in the studies carried out, the effects of the nanosolutions of the invention on the bacterial crown gall nematode (Meloidogyne sp.) were tested and it was investigated whether there were opportunities to fight this pest. Nanosolutions were stored at +4° C. until use. Bacterial crown gall nematodes used as target pests were collected as female individuals from the galls on infected plant roots in Bursa Karacabey tomato fields. Since the period that moves freely in the soil and infects the plant is the second period, 2nd period larvae were obtained from collected female individuals and experiments were carried out on 2nd period larvae. However, the species identification of bacterial crown gall nematode Meloidogyne was not made, it was expressed over the genus (Meloidogyne sp.).

The solutions were diluted in sterile distilled water to obtain the following doses: Solution #1: (2000 ppm liposomal ozone nanosolution) 50%, 25% and 12.5%, 2 Solutions 2 and 3: 0.1%, 0.2%, 0.3% and 0.4%.

Each solution was placed in sterile glass petri dishes with a diameter of 6 cm at the above doses, approximately 20 ml, and immediately after that, approximately 100 (±10) 2nd period nematode larvae were added to each petri dish. Afterwards, the petri dishes were shaken in a vortex for 1 minute and homogenization was achieved. Then, the petri dishes were covered and left for incubation at room temperature until the end of the experiment. As a control group, nematodes were kept only in sterile distilled water for the specified times.

The efficiency (toxicity) of the nanosolution was determined by counting the dead nematodes at the end of each solution trial at 6, 12 and 24 hours. Each experiment was carried out in 3 replications.

Solution 1, that is, nanosolution containing only “Ozone” in its content, tends to increase in its toxic effect as the exposure time on the nematode increases. However, there is no difference in efficacy between 12 hours and 24 hours. The lowest effect was detected after 6 hours and at the lowest dose of 12.5%, 25%, it was determined after 12 and 24 hours that it was effective on almost all nematodes at 50% dose, that is, it had a toxic effect. These 3 results show that solution 1 has a promising effect in combating Meloidogyne sp.

Solution 2, that is, nanosolution containing “Ozone+Acetic Acid”, tends to increase in its toxic effect as the exposure time on the nematode increases. However, there is no difference in efficacy between 12 hours and 24 hours. While the lowest effect was detected at the rate of 35% at the lowest dose of 0.1% after 6 hours, it was determined that it had a toxic effect on almost all nematodes at 0.3% and 0.4% doses after 12 and 24 hours. This result shows that solution 2 has a promising effect in combating Meloidogyne sp.

Solution 3, that is, nanosolution containing “Ozone+Acetic Acid+Thymol”, tends to increase in its toxic effect as the exposure time on the nematode increases. However, there is no difference in efficacy between 12 hours and 24 hours. While the lowest effect was detected at the rate of 42% at the lowest dose of 0.1% after 6 hours, it was determined that it had a toxic effect on almost all nematodes at 0.3% and 0.4% doses after 12 and 24 hours. This result shows that solution 3 has a promising effect in combating Meloidogyne sp.

As a result, increasing synergetic toxic effect of nanosolutions with 3 different ingredients on bacterial crown gall Meloidogyne sp. nematode has been shown. It has been demonstrated that the liposomal ozone nanosolutions of the invention have a high potential to suppress harmful nematodes, especially in the fight against this nematode in greenhouses.

Laboratory Test on Toxic Effect of Liposomal Ozone Nanosolutions on Two-Spotted Spider (Tetranychus Urticae (Koch) (Scary: Tetranychidae):

In this test, 10-week-old seedlings of eggplant (Pala variety) [Solanum melongena L.(Solanaceae)] plants, which is the plant species most damaged by the spider mite, were used as test material. Seedlings were grown from seed in a sterile climate room with artificial lighting (16 hours light: 8 hours dark) at 27t±1 and 60±5% relative humidity.

Two-spotted spider mites (Tetranychus urticae Koch) (Acari: Tetranychidae) individuals used in the experiment were recruited from a sensitive population collected from Bursa 8 years ago and grown continuously without pesticides. Colonies of the pest were reproduced on the same eggplant seedlings under controlled conditions.

Liposomal ozone nanosolutions of the invention were used in these tests. Two different (I and II) concentrations of two different formulations (N and S) were used in the first experiment. In the second experiment, combined formulations in which different concentrations of acetic acid (S+AA), orange oil (S+P), thyme oil (S+K) or lavender oil (S+L) were mixed to the I concentration of the S-coded formulation were tested.

The same volume of liposomal ozone nanosolutions was sprayed on and under the leaves of eggplant seedlings using hand sprays. As a control group, only one group was sprayed with water. Afterwards, the plants were kept in laboratory conditions for 30 minutes to dry the water and solutions applied to the leaves. 5 adult female (according to the economic damage threshold determined for eggplant) were placed on each leaf one by one with the help of a brush. Each experiment was carried out in 3 replications. Plants were kept in a sterile climate room with artificial lighting (16 hours light: 8 hours dark) at 27t±1 and 60±5% relative humidity for 7 days. At the end of the third 4th and 7th days, the living biological stages of the spider mite (egg, larva, and nymph, adult) were counted one by one under the stereomicroscope and noted.

The numbers of viable spider mites seen in eggplants in the water-only control group and the number of viable individuals on the eggplant leaf in which different formulations were applied were included in the Abbott formula (Abbott 1925).

According to the results of the tests performed under laboratory conditions on the 3rd day, concentrations I and II of the N formulation showed toxic effects respectively as follows; 80% and 93% on the eggs; 100% and 47% on larvae; 55% and 0% on nymphs and 46% and 53% on adults. The same concentrations of S formulation showed toxic effects respectively as follows; 30% and 93% on the eggs; 66% and 94% on larvae; 0% and 0% on nymphs and 8% and 69% on adults.

According to the results of the tests performed under laboratory conditions on the 7th day, concentrations I and II of the N formulation showed toxic effects respectively as follows; 46% and 73% on the eggs; 23% and 60% on larvae; 68% and 95% on nymphs and 14% and 71% on adults. The same concentrations of S formulation showed toxic effects respectively as follows; 5% and 0% on the eggs; 0% and 30% on larvae; 76% and 19% on nymphs and 15% and 29% on adults.

According to the results of the tests performed under laboratory conditions on the 3rd day, formulations in which a single formulation is combined with thyme oil (S+K), lavender oil (S+L), orange oil (S+P) and acetic acid (S+AA) to a concentration of 1000 mg/L showed toxic effects respectively as follows; 97, 99, 99 and 100% on the eggs; 95, 100, 100 and 100% on larvae; 0% on nymphs and 100, 100, 100 and 100% on adults.

According to the test results on eggplant plants under laboratory conditions, the inventive liposomal ozone nanosolutions, after three days, killed the eggs and larvae of the two-spotted spider mite at a rate of 30-93% and 47-100%, adults at rates of 8-69%. Results were not considered as nymphs did not occur in the first 3 days. Effects ranged from 19-76% as nymph populations naturally increased on day 7 of the test. Accordingly, different formulations and concentrations of the invention showed varying degrees of toxic effect on all biological stages of the spider mite. According to the best results, lethality (toxic effect) was observed up to 93% of eggs, 100% of larvae, 76% of nymphs and 69% of adults. The addition of plant-based oils to the formulations greatly increased the toxic effect on the spider mite and showed a synergistic effect. Even the lowest concentration (concentration no. I) of the nanosolution of the invention which is combined with thyme, lavender, orange oil or acetic acid showed toxic effect 97-100% to eggs, 95-100% to larvae; showed 100% on adults within three days. Since these mixtures showed a rapid effect within 3 days, they caused a very high rate of death of spider mites without allowing nymphs to form. As a result, it showed a toxic (poisonous) effect to the Two-spotted spider mite depending on the formulations and concentrations of the invention. The potential of using the inventive liposomal ozone nanosolution formulations as a Plant Protection Product in Plant Production stages in Agriculture has been demonstrated with this study.

Field Tests on the Toxic Effect of Liposomal Ozone Nanosolutions on Red Spider:

4 decares of land in Çeşmealti village of Çanakkale Biga district where farmer Halil Ba\check{g}cio\check{g}lu planted tomato seedlings that came early in the 80-85 days harvest on May 1 was divided into 100 square meters plots, the above table has been reached in the form of dose and mixture trials of the drugs that have been made and applied.

As of May 4, the plots in the form of doses and mixtures in the table were examined for red spider, early leaf blight from the seedling period. The plots were sprayed at intervals of 10-12 days and controls were made at intervals of 10-11 days by adding Lambda-cyhalothrin against powdery mildew from flowering period and green worm after fruit setting period.

In the observations made on Friday, May 15, no red spider pest, early leaf blight, powdery mildew and green worm have been encountered as of the seedling period.

In the observation made on Tuesday, May 26, it has been observed that the red spider population in the plots is 2-3 per m² where the drug is used in doses of 75-100 ml A (33% ozone emulsifier, 34% ozone oil, 33% acetic acid) and drug B (30% ozone emulsifier, 30% ozone oil, 30% acetic acid, 10% functional oil thymol), in the drug A and drug B plots, which were applied at a dose of 50 ml, 7-8 live red spiders were seen per m², and this was also found to be in a low population. Early leaf blight, powdery mildew and green worm have not been encountered since the seedling period. When we examined the green parts, no green worm eggs and larvae were found.

In the observation made on Tuesday, May 5, it has been observed that the red spider population in the plots is 4-5 per m² where the drug is used in doses of 75-100 ml A (33% ozone emulsifier, 34% ozone oil, 33% acetic acid) and drug B (30% ozone emulsifier, 30% ozone oil, 30% acetic acid, 10% functional oil thymol), in the drug A and drug B plots, which were applied at a dose of 50 ml, 8-9 live red spiders were seen per m², and this was also found to be in a low population. It was observed that there was no effect of drug A and drug B when compared with other drugs used against early leaf blight and powdery mildew at all doses used alone in plots. When all the plots were observed, when we examined the green parts, it was observed that there was a green worm egg and larva-dominated contamination with an average of 1 per 100 plants. Spraying will be done when there is 5% contamination against the green worm.

In the observation made on Tuesday, June 16, it was observed that the red spider population was 4-5 live per m² with the continuation of favorable weather conditions in the drug A and drug B plots used in 75-100 ml doses, It was determined that the red spiders in the drug A and drug B plots, which were applied at a dose of 50 ml, were 9-10 per m².

According to the observations in the plots, when compared with the other drugs used against early leaf blight and powdery mildew at all doses of drugs A and B used alone, it was observed again that there was no effect. The green parts of the plants in the whole plot were examined and dead green worm larvae were found from place to place. In drug A and drug B plots of dead green worm larvae, death occurred with an average of 1-2% at a dose of 50 m, it was observed that the rate of dead in the drug A and drug B plots applied at a dose of 75-100 ml was 3-4%. It was observed that the mixtures with lambda-cyhalothrin were better in terms of performance compared to drug A and drug B used alone.

In the observation made on Friday, June 26, it was observed that the red spider population was 3-4 live per m² with the continuation of favorable weather conditions in the drug A and drug B plots used in 75-100 ml doses, It was determined that the red spiders in the drug A and drug B plots, which were applied at a dose of 50 ml, were 7-8 per m². As a result of the examination of the green parts, 2 live green worm eggs and larvae were found in 100 plants.

| | | | | Drug A + | | | Drug B + |
|---|---|---|---|---|---|---|---|
| | | | | Abamectin + | | | Abamectin + |
| | Phytotoxic | | Drug A + | Lambda- | | Drug B + | Lambda- |
| | Dose | | Abamectin + | cyhalothrin + | | Abamectin + | cyhalothrin + |
| | Drug B | Drug A + | Early | Early | Drug B + | Early | Early |
| Control | 200 ml | Abamectin | leaf blight | leaf blight | Abamectin | leaf blight | leaf blight |
| Phytotoxic | Drug B | Drug A + | Drug A + | Drug A + | Drug B+ | Drug B + | Drug B + |
| Dose | 100 ml | Lambda- | Abamectin + | Abamectin + | Lambda- | Abamectin + | Abamectin + |
| Drug A | | cyhalothrin | Ashing | Lambda- | cyhalothrin | Ashing | Lambda- |
| 200 ml | | | | cyhalothrin + | | | cyhalothrin + |
| | | | | Ashing | | | Ashing |
| Drug A | Drug B | Drug A + | Drug A + | Drug A + | Drug B + | Drug B + | Drug B + |
| 100 ml | 75 ml | Abamectin + | Abamectin + | Abamectin + | Early leaf | Lambda- | Abamectin + |
| | | Early | Lambda- | Early | blight | cyhalothrin + | Early |
| | | leaf blight | cyhalothrin + | leaf blightv | | Early | leaf blight + |
| | | | Early | Ashing | | leaf blight | Ashing |
| | | | leaf blight | | | | |
| Drug A | Drug B | Drug A + | Drug A + | Drug A + | Drug B + | Drug B + | Drug B + |
| 75 ml | 50 ml | Ashing | Lambda- | Abamectin + | Ashing | Lambda- | Lambda- |
| | | | cyhalothrin + | Lambda- | | cyhalothrin + | cyhalothrin + |
| | | | Ashing | cyhalothrin + | | Ashing | Early |
| | | | | Early | | | leaf blight + |
| | | | | leaf blight + | | | Ashing |
| | | | | Ashing | | | |
| Drug A | Control | Drug A + | Drug A + | Drug A + | Drug B + | Drug B + | Drug B + |
| 50 ml | | Abamectin + | Early leaf | Abamectin + | Abamectin + | Early leaf | Abamectin + |
| | | Lambda- | blight + | Lambda- | Lambda- | blight + | Lambda- |
| | | cyhalothrin | Ashing | cyhalothrin + | cyhalothrin | Ashing | cyhalothrin + |
| | | | | Ashing + | | | Ashing + |
| | | | | Early leaf | | | Early leaf |
| | | | | blight | | | blight |

Content A: Ozonated oil + ozonated polysorbate 80 and Acetic acid B Contains ozonated oil + ozonated polysorbate 80 + acetic acid + thyme oil.

As a result of the general observations in the trial plots we made in the field of farmer Halil Ba\check{g}cio\check{g}lu in Çeşmealti village of Çanakkale Biga district, Drug A and drug B showed a performance of 35-40% in 50 ml alone and 70-75% in 75-100 ml in red spider. It was observed that the expected performance was even better in the experimental plots observed with the addition of Abamectin to Drug A and Drug B. In the trial plots, it was determined that drugs A and drug B had no effect against early leaf blight and powdery mildew, compared to the other drugs we observed, between the dates we observed. It was determined that the drug A and drug B used in the experimental plot showed 10-15% success in 50 ml and 30-35% in 75-100 ml against green worms. It was observed that the performance against green worm was better in the experimental plots observed with Lambda-cyhalothrin added to Drug A and Drug B. It was also observed that there was no problem in the drug mixtures in the table.

Investigation of the Efficacy of Liposomal Ozone Nanosolutions Against Nosema apis and Nosema cerenae Infections in Adult Honey Bees:

Nosema apis and Nosema cerenae cause Nosemosis in adult honey bees (Apis mellifera) and these factors settle in the digestive system of adult bees. Nosemosis is one of the most common bee diseases and causes significant bee losses worldwide. This disease causes digestive system disorders, decreased average lifespan of bees, colony number, honey production, pollen collection and significant winter losses in the colony. Nosemosis can progress with bacterial, protozoan and viral diseases, which adversely affects bee colony health, bee products and production. Various active substances have been used in the treatment of nosemosis until today, the most widely used of them is Fumagillin (Commercial name: Fumidil-B). However, this active ingredient is banned today because it poses a residue problem in honey. For this reason, treatment trials with organic acids, natural plant extracts and similar substances have been made in recent years. In this study, the efficacy of essential oils such as Origanum minutiflorum (Timol) and Artemisia absinthium (Wormwood) extract and Liposomal Ozone Nanosolutions against Nosemosis were investigated. Day 0 counts were made before the combinations created in this study were applied, sugar syrup used to feed bees was given to control groups in field trials. In the experiments, the combinations were mixed with sherbet (used to feed the bees) and applied to the sherbet inside the hive or to the frames by spraying. 50 bee samples were taken from each hive from the apiary and the protocol number was given before starting the experiments. After the samples were kept in the freezer for one day, the digestion method was applied for the control of positiveness and negativeness in terms of Nosema spores. In this method, 10 bee samples were taken from each hive and the abdomens of the bees were separated from their bodies with the help of a scalpel. 1 ml per abdomen, 10 ml in total distilled water was added. Abdomen were crushed in a suitable beaker, a drop of abdominal fluid was transferred to a Neubauer Toma slide and Nosema spores were investigated at 40×10 magnification. In order to determine the spore load of Nosema fungus by the digestion method mentioned above in the positive hives, 20 bee samples were taken and the Nosema agents were counted under the microscope. While the experimental groups were formed with a certain amount of hives for each combination, it was noted that the Nosema spore number averages between the experimental groups were close to equal. The combinations were administered orally by spraying into the hives or by placing them in the sherbet inside the hive. 20 bee samples were collected from each hive at certain day intervals and Nosema spore counts were made by digestion method. Ozone (Liposomal Ozone Nanosolution), Thymol and Artemisia absinthium extracts and their combinations were tested in 5 different concentrations and dosages. According to the efficiency rates of the three combinations found most effective in these five field trials; Ozone+Thymol (Spray): 2000 ppm 200 ml Ozone+100 ml 3% Thymol+700 ml sugar syrup 89.47%, Timol+Artemisia (Oral): 250 ml 2% Thymol+200 ml 2% Artemisia absinthium+550 ml sugar syrup 85.95%, Ozone (Oral): 100 ml 1000 ppm Ozone+400 ml sugar syrup 75.08% were given respectively. This result supported the previous studies with Timol, and when the combination was enriched with nanoparticle ozone, the results were found to be more effective against Nosema. There were no deaths or side effects caused by the application.

It has been determined that the antioxidant effect of ozone in nanocarrier systems is higher than the oils with larger molecules, and it shows its antibacterial effect at lower doses in the biocompatibility tests performed within the scope of the invention, It was understood that ozonated emulsifiers showed toxic effects in cytotoxicity tests performed at these doses. Therefore, in a different embodiment of the invention, ozonated emulsifiers are coated with biocompatible materials. In this way, in addition to the solutions created by the transportation of ozone with oils or emulsifiers containing oil and diversified by the addition of substances such as oil, acid, vitamin and mineral, it is possible to obtain new nanocarrier structures that provide high stability.

Coating of ozonated emulsifier is important for reducing the aggressive behavior of active ozone, providing slow release, obtaining small particle size and complying with sterilization methods. Ozone cannot be sterilized by heat and there is no sterilization method other than filtration. In this sense, it is necessary to realize coatings suitable for filtration and to choose suitable coating materials.

In the most basic application of the invention, at least one emulsifier and ozone are used. Polysorbates such as polysorbate 20, polysorbate 60, polysorbate 80, polysorbate 85 can be used individually or in combination as emulsifiers. In another embodiment of the invention, dosage can be made by adding water (with or without mixing) to the ozonated emulsifier mentioned.

In a preferred embodiment of the invention, the ozonated emulsifier is coated with at least one emulsifier and/or polymer. The ozonated emulsifier and the emulsifier coated on it can preferably be polysorbate 20, polysorbate 60, polysorbate 80, polysorbate 85 or a combination thereof. The ozonated emulsifier and the emulsifier coated on the ozonated emulsifier can be the same or different polysorbates. For example, the structure obtained with polysorbate 80, and ozone can be coated with polysorbate 20. This structure may contain water. It is possible to use different polymers as a polymer, depending on the purpose and area of application.

Different polysorbates can be used in the invention, but not limited to those mentioned herein. Polysorbate 80 contains unsaturated fatty acid in the tail. Ozonated polysorbate 80 is formed by ozonation of this fatty acid. Polysorbate 85 carries 3 unsaturated fatty acids in its tail. Therefore, more ozone can be loaded.

In another embodiment of the invention, polyethylene glycol (PEG) and glycerin can be used as biocompatible materials for the coating. Ozone emulsifiers can be coated with glycerin alone, with PEG alone, or with a combination of glycerin and PEG. Coating ozonated emulsifiers with glycerin and PEG slows down the rapid antioxidant effect. Coating the ozonated emulsifier with polymers in addition to PEG and/or glycerin contributes to increased stability. Ozonated structures in the form of nanocarriers have two-year stability and have been found to maintain their antibacterial activity. Coating ozonated emulsifiers with glycerin and/or PEG provides a stability-enhancing effect within the scope of the invention However, coating with mannitol and/or dextran is also possible. The ozone emulsifier, coated with glycerin and/or PEG, reaches the target organ/tissue without the ozone gasification and, after reaching, it is possible to get rid of said coating and use it in tissue. For example, in the case of use in cancer treatment, cancer target protein or other cancer drugs are bind to the ozone emulsifier and this new formation is coated with glycerin and/or PEG. The coating dissolves rapidly and the target protein binds to the cancer cell in the target tissue. In this way, ozone is dissolved and the cancer cell is broken down. This applies to all other chemicals.

The dimensions of ozonated emulsifiers are approximately 20 nanometers. Small coated nanoliposomes are formed by simply coating this molecule with PEG. Slightly larger nanoliposomes are obtained by coating with glycerin. Particle sizes increase when other chemicals are added to these nanoliposomes. It is important in drug formation that the products remain below 200 nanometers in these measurements The amount of coating and the ratio of glycerin and/or PEG are also important in terms of intended use and size. If the coating is done only in small quantities with PEG, the particles grow minimally. If the ratio is increased or the amount of glycerin is increased, the particle size increases.

When ozonated polysorbate 80 is first coated with glycerin and then with PEG, a 13-nanometer solution is obtained when the new mixture is mixed with water. Some glycerin and PEG that are not added to the complete mixture are also seen in the measurement. It ensures liposomes to be sent to the target organ without causing damage to the tissues and have a slower effect by using it in this form

In order to increase the effectiveness of ozone emulsifiers, it is possible to bind the lipoproteins, some of which are in lipid structure, to the surfactant so as to send them to the appropriate target organ. The lipoprotein-coupled structure is coated with glycerin and PEG. Similarly, phospholipid, sphingolipid, ceramide, proteolipid, glycolipid, isoprenoids, phosphoglyceride, lipids and terpenes, mineral, protein, cancer target protein, DMSO, acetyl cysteine, hyaluronic acid, menthol, acids, alcohols, enzymes such as ogenase, elastase, trypsin, lipase, alkans can be bind individually or in combinations to ozone emulsifiers.

Alternatives that can be used in different applications of the invention and the technical effects provided by them are explained below. The smallest form of the solutions obtained is referred to as niosome in the literature. These are the smallest liposome structures made with surfactants only in the smaller form of liposomes.

After obtaining the smallest structure, it is aimed in the invention to develop products with increased stability and reduced toxicity by using different components. Accordingly, in another application of the invention, in addition to ozone and emulsifier, the structure containing ozone or non-ozone phospholipid is coated with glycerin and/or PEG. It is especially found in the sphingomyelin nerve cell. The aim of this structure; it is used to make selective drugs for nerve cells when given to the body, Said structure adheres to nerve cells and functions to repair the nerve cell with ozone. In this way, it is possible to play a role in the repair of nerve cells, such as the use of ozone in wound healing.

In another application of the invention, in addition to ozone and emulsifier, the structure containing ozone or non-ozone sphingolipid is coated with glycerin and/or PEG. In this way, nano liposome that selectively adheres to the target organ for again nerve cells are obtained.

In another application of the invention, in addition to ozone and emulsifier, the structure containing ozone or non-ozone proteolipid is coated with glycerin and/or PEG. In this way, nano liposomes that selectively adhere to the target organ for nerve cells are obtained.

In another application of the invention, in addition to ozone and emulsifier, a glycolipid containing structure with or without ozone is coated with glycerin and/or PEG. This structure functions as a nanoliposome, which selectively adheres to the target organ for nerve cells. Cerebrosides, Ceramide oligosaccharides, Sulfatides, Gangliosides can be used in combination for ozone carrier nanoliposomes suitable for use according to their target organs.

In another application of the invention, in addition to ozone and emulsifier, the structure containing lipoproteins with or without ozone is coated with glycerin and/or PEG. The nanoliposome HDL cholesterol target organ is the liver, which is attached to the target organ by combining lipids such as cholesterol. Cholesterol circulating in the blood goes to the liver last. It is used in the treatment of liver diseases and in liver cancer in combination with ozonated emulsifier and HDL cholesterol.

In another application of the invention, in addition to ozone and emulsifier, the structure containing isoprenoids is coated with glycerin and/or PEG. For example, a combination of vitamins such as fat-soluble vitamins, vitamin A can be given.

In another application of the invention, in addition to ozone and emulsifier, the structure containing ozone or non-ozone ester phosphaglycerides is coated with glycerin and/or PEG. As an example, Phosphatidylethanolamine (kephalin, cephalin) can be given. Cephalins are esters of phosphatidic acid that function in the brain. Since it is abundant in the brain as a target organ, it plays an active role in the transmission of ozone. Lecithin is the main constituent in many tissues. Phosphotidylserine is found in blood coagulation; phosphatidylinositol is intracellular messenger; phosphatidyl glycerol is found in Cardiolipin in cardiac cells; Malignolipin is found in malignant cells. It is possible to perform target organ treatments with the combination of these lipid derivatives.

In another application of the invention, in addition to ozone and emulsifier, the structure containing minerals is coated with glycerin and/or PEG. Nano liposomes formed by the combination of metal ozone formed in this group are obtained. The combination formed in this group is directed to the target organs again It facilitates the penetration of ozone into the cancer cell by opening the channels with the use of magnesium so as to open the calcium channels of the cell in cancer treatment. Elements such as zinc, copper, sulfur and silver also increase the antibacterial properties of the combination and are used in different target organs. For example, in agriculture, leaf health can be used as nanoliposome to increase plant health.

In another application of the invention, in addition to ozone and emulsifier, the structure containing DMSO is coated with glycerin and/or PEG. DMSO is selective for cancer cells with its combined use. In this way, ozone is transported to the target organ.

In another application of the invention, in addition to ozone and emulsifier, the structure containing ozone or non-ozone vegetable oils is coated with glycerin and/or PEG. In this way, nano liposomal and liposomal structures formed for the transport of ozone to the tissue are obtained.

In another application of the invention, in addition to ozone and emulsifier, the structure containing terpenes with or without ozone is coated with glycerin and/or PEG. It is used to form liposomes and nano liposomes of active oils secreted from plants. The effect of terpenes and at the same time the use of ozone as nano liposomes is possible with their combined use

Forms in different applications mentioned above or combinations of these forms can be used in the invention One or more combinations of antioxidants such as vitamins, minerals, dextran, mannan, chitosan, PLL and PEI polymers, protein, amino acid, sugar, sweetener, alcohol, acid, menthol, hyaluronic acid, tranexamic acid, N acetyl cysteine, anticancer drugs can be added to the combinations.

In a different application of the invention, after the ozone emulsifier and oils are coated with glycerin, a solution can be obtained by adding menthol and sucralose and finally coating with hyaluronic acid. Various viruses, including SARS Coy 2, can be transmitted by hand (skin). However, there is also the possibility of eye contamination. Therefore, biocompatibility tests were performed for the eyes and skin, with the determination of the antibacterial effects of the solution within the scope of the invention. In the solution suitable for use as a nasal spray, in addition to the ozone emulsifier, zinc is also used in the liposome with hyaluronic acid glycerin.

In a different application of the invention, ozonated or unozonated phospholipid, cholesterol, sphingolipid, ceramide, proteolipid, glycolipid, isoprenoids, phosphoglyceride, lipids and terpenes, minerals, protein, cancer target protein, silicon dioxide, organic silicon, DMSO, acetyl cysteine, vitamins, hyaluronic acid, menthol, acids, alcohols, enzymes such as collagenase, elastase, trypsin, lipase, alkanes, protein, amino acid, sugar, sweetener and/or tranemic acid are added to the ozone emulsifier so as to form the main structure. In order to coat said main structure, coating is made of glycerin, mannitol, dextran and/or PEG and polymer.

There are two preferred main applications of the invention. These are the injection formula and the spray formula. Cholesterol can be added to ozonated polysorbate in the injection formula. In this formulation, polysorbate coating and mannitol, glycerin, dextran, PEG, hyaluronic acid alone or combinations can be coated on it. In the spray formula, ozonated sunflower oil, glycerin, menthol, hyaluronic acid and zinc can be added to ozonated polysorbate. It is also possible to make different variations of this formulation. Nasal spray and mouth spray contain all of the ingredients mentioned. However, wound spray and eye drops only contain ozonated polysorbate, glycerin, hyaluronic acid and zinc.

The usage areas and usage forms of the invention are listed below:
In the medical and veterinary fields, it can be used by way of intravenous, intra-arterial, intra-articular, subcutaneous, intramuscular, intraperitoneal, intra-bladder, intrauterine, rectal, inhaler, ear canal, nasal passage, oral route, eye drops, in vitro fertilization and oocyte sperm development and can be used in culture enhancing treatments.
It can be used in disinfectant, antibacterial paint, antibacterial fabric, wound dressing in the field of chemistry.
It can be used to increase the performance of fuels.
It can be used in agriculture for plant health-enhancing, plant-enhancing disinfectant, insecticide, repellant, seed improvement and irrigation.
It can be used as livestock disinfectant, skin protector, by way of intra-arterial, intra-articular, subcutaneous, intramuscular, intraperitoneal, bladder, intrauterine, rectal, ear canal, nasal passage, oral, also as eye drops.
It can be used in textile fabric bleach and antibacterial fabrics.
It can be used as a gasoline additive.
It can be used as food raw material, food preservative and food additive.
It can be used as skin rejuvenation and hair developer in cosmetics.
The products can be used in ampoules, vials, sprays, creams and solutions.

Trial results determining the effects of three different solutions (Farmoxyn 1, 2 and 3) applications produced in the studies carried out within the scope of the invention on the yield and quality of lettuce and tomato plants are given below. The aim of the experiment is to determine the effects of three different solutions (Farmoxyn 1, 2 and 3) on the yield and quality of plants while investigating the possibilities of using them in agriculture. In this experiment, it was investigated whether the solution applications named Farmoxyn 1, 2 and 3, which were made during the cultivation of lettuce and tomato plants, which are widely grown in our region, were compared with the control plants and whether they were effective on the yield and quality criteria. This research was carried out in Bursa Uluda\check{g} University Faculty of Agriculture, Department of Horticulture, Application and Research Greenhouse and End Harvest Physiology Laboratory between February 2021-June 2021.

1. Material

In this study, 2 different tomato varieties (Nazli F1 and Atakan F1) and lettuce (Lactuca sativa) belonging to the tomato (Lycopersicon esculentum L.) species were used as plant material.

2. Method

In the trial, three different solutions (Farmoxyn 1, 2 and 3), made by Biopharma Pharmaceuticals Chemistry Health Industry Trade. Ltd., were used. These doses are grouped as follows; Farmoxyn 1 (1), 0.2%; Farmoxyn 2 (2) is grouped as 0.2% and Farmoxyn 3 (3) 10%. The untreated seedlings were considered as the control group. Water treatments were applied to the control group plants at the same time as the other treatments.

2.1 Ozone Application

Tomato and lettuce seedlings were immersed into grouped solutions 1 (% 0.2); 2 (% 0.2) and 3 (% 10) just before planting in the greenhouse. 5 applications were made from planting to the beginning of harvest on tomato plants. On the other hand, 2 applications were made on leaf lettuce plants. Applications continued with an interval of 15 days in both plant groups. It was made as 100 mL (for each variety, each application dose) in tomato, in the process from fruit setting to harvest, 200 mL (for each variety, for each application dose) foliar spraying was applied for each application. On the other hand, in leaf lettuce, 100 mL (for each application dose) was applied as a foliar spray in each application.

2.2. Analysis and Measurements Made in Tomatoes

Fruit length (cm), fruit diameter (cm), color determination, water soluble dry matter (SSCM), titratable acid (TEA) and yield per plant (kg) of the samples taken from the beginning of the harvest were analyzed and measured.

2.3. Analysis and Measurements Made in Leaf Lettuce

Leaf length, root length, number of marketable leaves, number of non-marketable leaves, root length, root fresh weight, root dry weight, leaf fresh weight, leaf dry weight, leaf proportional water content, color determination, chlorophyll determination of the samples taken after harvest in leaf lettuce were measured.

3.1. Tomato Results

3.1.1. Fruit Length and Diameter

The best result for Nazli cultivar was obtained from the application number 1 and it was statistically different from other groups.

When the data of Atakan cultivar were evaluated, the best results in terms of fruit size were obtained from application groups 1 and 2 and they were in the same group statistically. In terms of fruit diameter, all application groups gave better results than the control group and were statistically included in the same group.

3.1.2. Determination of Color in Fruit

When the brightness values of Nazli F1 and Atakan F1 varieties are examined, the application group is statistically different from the other groups and gives the best result is the application number 1. The intensity of red color in tomato fruit is one of the most important criteria for determining the quality. When a value is evaluated, better results were obtained for Nazli variety in applications 1 and 3 compared to other application groups and statistical difference was determined. Application number 1 gave the best results for Atakan variety. This application is followed by application groups 2 and 3 and both are in the same statistical group. The lowest a values for both Nazli and Atakan cultivars were obtained from the control group. When b values were examined, the lowest value for Nazli variety was obtained from the application number 1. This value represents the yellow color. While the control group has the highest value, applications 2 and 3 fall into the same statistical group, although the lowest numerical value for Atakan cultivar was obtained from the application group number 1, no statistical difference was determined.

3.1.3. WSDM Amount

Statistically different and the best results in terms of WSDM (Water Soluble Dry Matter) were obtained from the application no. 1 in Nazli cultivar, Application groups 1 and 3 in Atakan cultivar gave the best results and entered the same statistical group.

3.1.4. TEA Amount

When TEA (Titratable acid) values were evaluated on the basis of variety, it was determined that the best result was found in application number 3 and the lowest result in application number 1. There was no statistical difference in Atakan variety.

3.1.5. Yield Per Plant (Kg)

Average yield was found by dividing the number of fruits per plant. The best result of Nazli cultivar was obtained from application group 1 with an average of 7.84 kg and a statistical difference was determined. Control and application group 2 are in the same statistical group and it was determined that lower yield was obtained than the yield obtained from number 1. In Atakan cultivar, although the number 1 application seems to be numerically superior with 6.68 kg, the application 2 is in the same statistical group with 6.48 kg. The control group and the application group no. 3 gave lower results than the application groups no. 1 and 2.

3.2. Leaf Lettuce Results

3.2.1. Leaf and Root Lengths

When all application groups were compared, the application group that gave the best results in terms of leaf and root length was number 1 and was statistically different from the other groups. Applications numbered 2 and 3 were included in the same statistical group. The control group, on the other hand, gave the lowest application result.

3.2.2. Wet and Dry Weights

Considering all weight parameters, the application group number 1 was statistically different from all groups and the best results were obtained.

3.2.3. Number of Marketable and Non-Marketable Leaves

The application that gave the best results in the study was 1 (33.55 units) and was statistically different from the other groups. Considering the number of non-marketable leaves, the lowest result was again obtained from the application no. 1 (3.04 units). As a result, there are more marketable and less non-marketable leaves in application number 1 compared to other application groups.

3.2.4. Leaf Proportional Water Content (%)

When LPWC (%) was examined, application groups 1 and 3 gave the best results and entered the same statistical group. The application no. 2 and the control group were included in different application groups from the application groups no. 1 and 3, and the lowest result was obtained from the control group. Thus, it was observed that application groups 1 and 3 were more resistant to stress conditions.

3.2.5. Leaf Total Chlorophyll (μmol/m2) Amount

When the results of the leaf chlorophyll amount were evaluated in the study, it was determined that the best application was number 1, followed by 2, 3 and control group applications

3.2.6. Leaf Color

As a result of the applications, it was determined that the best application in terms of brightness was the number 1 application and it was found to be statistically different. In the study, it was determined that the best result numerically was the number 1 application with the value of −19.14, however, it was determined that it is in the same statistical group with application number 3. It was determined that the salads in these treatment groups were greener than the other treatment groups. Considering the b values, although the number 1 application gave the best result in terms of numerical value, all of them were in the same statistical group, except for the control group.

In the final case, three different solutions included in the experiment were applied to the plants in tomato and leaf lettuce, and the obtained plants were harvested, and their quality parameters were examined. After the measurements and analyses, it was determined that 0.2% concentration of Farmoxyn 1 formulation had a positive effect on both types of vegetables.

1. A liposomal and/or niosomal ozone nanosolution comprising ozonated emulsifier.

2. The nanosolution according to claim 1, wherein the nanosolution comprises one or more that one of lecithin, lysophospholipid, polyethylene glycol, phosphatidylethanolamine, pluronic, polysorbates and a pharmaceutically acceptable emulsifier.

3. The nanosolution according to claim 1, comprising water comprising one or more than one of distilled water, salt water, sugar water, mineral water, deionized water, demineralized water, spring water, saline solution, physiological saline, and plant waters.

4. The nanosolution according to claim 1, comprising a carrier oil selected from the group consisting of: soybean oil, centaury oil, sesame oil, palm oil, poppy oil, soy lecithin, cholesterol, b-sterol, triglyceride, olive oil, fish oil, sunflower oil, castor oil, saffron oil, coconut oil, triglyceride derivatives, tributyrin, tricaproin, tricaprylin with paraffin, ethyl oleate, methyl oleate, or a combination thereof.

5. The nanosolution according to claim 1, comprising functional oil selected from the group consisting of: fixed oils, essential vegetable oils, or a combination thereof.

6. The nanosolution according to claim 1, comprising organic acid selected from the group consisting of: formic acid, phosphoric acid, hydrochloric acid, acetic acid, propionic acid, butyric acid, valeric acid, caproic acid, oxalic acid, lactic acid, malic acid, citric acid, benzoic acid, carbonic acid, phenol, uric acid, taurine, aminomethylphosphonic acid, or a combination thereof.

7. The nanosolution according to claim 1, comprising excipient selected from the group consisting of: anesthetics, pharmaceuticals pharmaceutical active substances such as water and/or fat soluble vitamins, minerals, hyarulonic acid, thymol, menthol, glycerin, ethyl alcohol, cetyl alcohol, butyl alcohol, benzyl alcohol, amino acids, acetyl cysteine, glutathione, herbal extracts, lidocaine, xylocaine, or a combination thereof.

8. The nanosolution according to claim 1, comprising 100 stock ppm-60 000 stock ppm ozone gas.

9. The nanosolution according to claim 8, comprising stable active ozone gas at doses of 1 ppm, 200 ppm, 500 ppm, 1000 ppm, 1600 ppm, 2000 ppm, 3000 ppm, depending on the application area.

10. The nanosolution according to claim 1, comprising liposomal ozone nanoparticles in sizes below 1000 nm, preferably below 200 nm, more preferably below 50 nm.

11. Liposomal ozone nanosolution production method according to claim 1, comprising the following process steps:
selecting an appropriate emulsifier and/or carrier oil for the application purpose,
weighing the emulsifier and/or carrier oil,
ozonation of emulsifier and/or carrier oil by passing ozone gas or nanobubble ozonated water through it,
weighting ozonated emulsifier and/or carrier oil again and determining the ozone content,
reduction of ozonated emulsifier and/or carrier oil to nanosize, and
preferably adding components selected from at least one excipient, at least one functional oil, at least one organic acid into nanosolution and mixing thereof.

12. (canceled)

13. (canceled)

14. (canceled)

15. (canceled)

16. (canceled)

17. (canceled)

18. The nanosolution according to claim 1, comprising at least one emulsifier, ozone and water.

19. The nanosolution according to claim 1, comprising:
one or a plurality of ozone emulsifiers,
at least one emulsifier and/or polymer-containing coating to cover said ozone emulsifier.

20. The nanosolution according to claim 19, one or more of polysorbate 20, polysorbate 60, polysorbate 80, and polysorbate 85 as emulsifier.

21. (canceled)

22. The nanosolution according to claim 18, comprising:
one or more ozone emulsifiers,
ozonated or unozonated phospholipid, cholesterol, sphingolipid, ceramide, proteolipid, glycolipid, isoprenoids, phosphoglyceride, lipids and terpenes, minerals, protein, cancer target protein, silicon dioxide, organic silicon, DMSO, acetyl cysteine, vitamins, hyaluronic acid, menthol, acids, alcohols, enzymes such as collagenase, elastase, trypsin, lipase, alkanes, protein, amino acid, sugar, sweetener and/or tranemic acid so as to form the main structure by adding the same to the ozone emulsifier, and
coating of glycerin, mannitol, dextran and/or PEG and polymer to coat said main structure.
